(19) United States
(12) Patent Application Publication
(10) Pub. No.: US 2003/0224000 A1
(43) Pub. Date: Dec. 4, 2003

(54) Methods for blocking or alleviating staphylococcal nasal colonization by intranasal application of monoclonal antibodies

(76) Inventors: John Fitzgerald Kokai-Kun, Frederick, MD (US); Jeffrey Richard Stinson, Brookville, MD (US); Scott Michael Walsh, Germantown, MD (US); Andrew Lees, Silver Spring, MD (US); James J. Mond, Silver Spring, MD (US); Gerald Walter Fischer, Bethesda, MD (US)

Correspondence Address: Finnegan, Henderson, Farabow,, Garrett & Dunner, L.L.P., 1300 I Street, N.W., Washington, DC, 20005-3315, US

(21) Appl. No.: 10/323,904

(22) Filed: Dec. 20, 2002

Related U.S. Application Data
(60) Provisional application No. 60/341,806, filed on Dec. 21, 2001.

(51) Int. Cl. A61K 39/40; A61K 38/16

(52) U.S. Cl. 424/165.1; 514/2

(57) ABSTRACT

This invention provides MAbs for blocking and alleviating nasal colonization by staphylococci and methods for their use in the anterior nares.

### CROSS-REFERENCE TO RELATED APPLICATIONS

This application is based on and claims the benefit of U.S. Provisional Application S. No. 60/341,806, filed Dec. 21, 2001 (Attorney Docket No. 7787.6003). The entire disclosure of this provisional application is relied upon and incorporated by reference herein.

### INTRODUCTION

Staphylococcal infections are a significant cause of morbidity and mortality, particularly in settings such as hospitals, schools, and infirmaries. Patients particularly at risk include infants, the elderly, the immunocompromised, the immunosuppressed, and those with chronic conditions requiring frequent hospital stays. Further, the advent of multiple drug resistant strains of Staphylococcus aureus increases the concern and need for timely blocking and treatment of such infections. Indeed, the recent World Health Organization report entitled “Overcoming Astionicro Oral Resistance” detailed its concern that increasing levels of drug resistance are threatening to erode the medical advances of the recent decades. Among the issues raised are infections in hospitalized patients. In the United States alone, some 14,000 people are infected and die each year as a result of drug-resistant microbes acquired in hospitals, so called nosocomial infections. Worldwide, as many as 60% of hospital-acquired infections are caused by drug-resistant microbes.

In infections caused by S. aureus, it appears that a principal ecological niche and reservoir for S. aureus is the human anterior nares. Nasal carriage of staphylococci plays a key role in the epidemiology and pathogenesis of infection (13, 22, 31, 48, 66, 69, 70, 72). In healthy subjects, three patterns of S. aureus nasal carriage can be distinguished over time: approximately 20% of people are persistent carriers, approximately 60% are intermittent carriers, and approximately 20% apparently never carry S. aureus (31).

Nasal carriage of staphylococci is an important risk factor for contracting S. aureus infection. Patients at greatest risk are those undergoing inpatient or outpatient surgery, in the Intensive Care Unit (ICU), on continuous hemodialysis, with HIV infection, with AIDS, burn victims, people with diminished natural immunity from treatments or disease, chronically ill or debilitated patients, geriatric populations, infants with immature immune systems, and people with intravascular devices (13, 22, 24, 31, 32, 38, 48, 70, 72). In one study of ICU patients (18), it was found that on admission 166 of 752 (22%) of patients were S. aureus nasal carriers. The probability of developing a staphylococcal infection was significantly greater (p<0.0001, with a relative risk of 59.6) in these patients than in non-carriers. In 28 out of 30 cases of subsequent staphylococcal infection, identity was found between the S. aureus strain colonizing the nares and the strain isolated from the infection. Even more strikingly, Mest et al. (42) showed that, of 19 patients who were admitted to the ICU with positive nasal cultures for S. aureus, 5 (26%) subsequently developed staphylococcal infections as compared to only 6 S. aureus infections in a group of 465 patients (1.3%) negative for nasal carriage of staphylococci.

Chang et al. (12) studied 84 patients with cirrhosis admitted to a liver transplant unit. Overall, 39 (46%) were nasal carriers of S. aureus and 23% of these patients subsequently developed S. aureus infections as compared to only 4% of the non-carriers. A study of HIV patients (48) showed that 49% (114 of 296) of patients had at least one positive nasal culture for S. aureus. Thirty four percent of 201 patients were considered nasal carriers, with 38% of these being persistent carriers, and 62% intermittent carriers. Twenty-one episodes of S. aureus infection occurred in thirteen of these patients. Molecular strain typing indicated that, for six of seven infected patients, the strain of S. aureus isolated from the infected site was the same as that previously cultured from the nares. The nasal S. aureus carrier patients were significantly more likely to develop S. aureus infection (P=0.04; odds ratio, 3.6; attributable risk, 0.44). This finding led the authors to conclude that nasal carriage is an important risk factor for S. aureus infection in HIV patients (48).

As discussed above, antibiotic resistance continues to be a major problem in staphylococcal infections and the anterior nares is a primary ecological niche for these strains as well. Methicillin resistant S. aureus (“MRSA”) is a well-documented public health problem. In one study performed in a nursing home, 29% of the residents carried S. aureus in the nares and, of those isolates, 31% were MRSA (34). In a separate study of postoperative intra-abdominal infection, it was concluded that MRSA may beta causative pathogen in postoperative intra-abdominal infection and this may be related to nasal colonization (22).

Current technology uses mupirocin ointment to clear staphylococcal nasal colonization. Indeed, antibiotics like mupirocin have been successfully used as intranasal antimicrobial agents in the eradication of nasal carriage of both methicillin sensitive and resistant strains of S. aureus (21, 32, 38, 62, 70). However, mupirocin resistant strains of S. aureus are emerging in many different geographical areas (14, 17, 19, 37). Therefore, based on these considerations, there is a need in the art for a non-antibiotic intervention to block or alleviate nasal carriage of S. aureus and other staphylococci. Particularly, there is a need in the art for an intervention that is immediate and directed to the mammalian nares.

### BRIEF DESCRIPTION OF THE INVENTION

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate embodiments of the invention and together with the description, serve to explain the principles of the invention.

This invention relates to the administration of monoclonal antibodies (MAbs) to those at particular risk for the complications of staphylococcal infections for the purpose of blocking or alleviating staphylococcal nasal colonization. Populations at risk include the very young, the very old, patients admitted to the hospital for in-patient or out-patient surgical procedures, patients suffering from various conditions that predispose them to staphylococcal infections, or any patient prior to release from a hospital. The use of MAbs as a pre-release treatment will serve to inhibit community spread of hospital-acquired staphylococcal strains. Administration of the MAbs of the invention may have multiple beneficial effects including alleviation of pre-existing staphylococcal nasal colonization and blocking of staphylococcal nasal colonization. MAbs of the invention can also be used as part of a comprehensive infection control program to reduce or prevent MRSA nasal colonization in a population and thus spread and subsequent disease.

As noted above, the anterior nares are a primary reservoir for staphylococci, and a strong correlation has been demonstrated between staphylococcal nasal colonization and subsequent staphylococcal infections in colonized individuals. It may also be possible to spread nasal colonization or even staphylococcal infections to individuals near those who are colonized. This invention blocks and/or alleviates staphylococcal nasal colonization in colonized individuals, thereby reducing the chance of subsequent infection in treated individuals. The invention may also be used to block or alleviate colonization of epithelial cells throughout the body. Moreover, the reduction of colonization in individuals reduces the overall frequency of staphylococcal infections in the general population. Global reduction of staphylococcal colonization in a community is especially important given the emergence of antibiotic-resistant staphylococcal strains, such as MRSA. Decreasing the number of new staphylococcal infections, by decreasing nasal colonization, in turn decreases the rate at which new resistant strains appear in the general population.

The invention includes methods of using both single MAbs and combinations of MAbs to alleviate and/or block S. aureus colonization of the anterior nares. The MAbs of the invention include anti-lipoteichoic acid MAbs, anti-peptidoglycan MAbs, and MAbs specific for other staphylococcal antigens, and modifications of these MAbs. These modifications include Fc mutants of these MAbs that contain identical antigen binding sites but modified Fc regions. The invention also includes chimeric MAbs specific for staphylococcal antigens, including those listed above, and methods for their use. In one embodiment, these MAbs are administered into the nares of normal or nasally colonized human subjects or other mammals to block or alleviate staphylococcal colonization of the anterior nares. Such treatment is not only beneficial to the colonized individual but also reduces staphylococcal reservoirs in the general population, thus reducing subsequent staphylococcal infections and limiting the spread of drug resistant, S. aureus as discussed above. Thus, administration to all or a portion of a patient population, for example, hospitalized patients, healthcare providers, pigs, cattle, sheep, goats, or other herded animals, may increase the overall health of the population.

### BRIEF DESCRIPTION OF THE DRAWINGS

FIG. 1 shows a schematic diagram of plasmid pSUN29, which contains the human IgG1 constant region.

FIG. 2 shows the amino acid (SEQ ID NO: 1) and nucleotide (SEQ ID NO: 2) sequences of the mutated human IgG1, which is cloned into plasmid pSUN30. Amino acid point mutations are shown in bold.

FIG. 3 shows a schematic diagram of the bicistronic expression plasmid pSUN31, which expresses the human/mouse chimeric anti-LTA Fc mutant monoclonal antibody, A110 Fc.

FIG. 4 shows the results of the antibody production ELISA for MAbs A110 (chimeric 96-110) and A110 Fc (CH3 mutant).

FIG. 5 shows the results of the activity ELISA for MAbs A110 (chimeric 96-110) and A110 Fc (CH3 mutant).

FIG. 6 shows the effect of different carriers, chitosan and polystyrene sulfonate, on retention time of antibodies in the anterior nares.

FIG. 7 shows that increasing chitosan (CS) concentration did not increase the retention of MAb in the nose.

FIG. 8 shows the effect of polystyrene-Ab microsphere size on nasal MAb retention.

FIG. 9A and FIG. 9B shows that salt concentration and type affects the encapsulation efficiency of MAb in the microspheres.

FIG. 10 shows the effect of polystyrene sulfonate molecular weight on MAb retention.

FIG. 11 shows that cream formulations alone or in combination with mucoadhesive polymers prolong nasal retention of MAbs in a similar manner as mucoadhesive polymers alone.

### DETAILED DESCRIPTION OF THE INVENTION

One aspect of the invention is directed to a method for combating staphylococcal infections by administering to the mammalian nares MAbs directed to antigens of staphylococci to block or alleviate colonization of the nares by staphylococci. In another aspect of the invention, anti-LTA MAbs may be used to block or alleviate adherence to, colonization of, or infection of epithelial cells at sites throughout the body. These sites include the nose, the skin, the eyes, the mouth, and the respiratory track. The MAbs may be administered either singularly or in combination.

The term “antibody,” as used herein, includes full-length antibodies and portions thereof. An antibody has four polypeptide chains, two light chains and two heavy chains. Each chain is divided into two regions, the variable region (which confers antigen recognition and binding) and the constant region (associated with localization and cellular interactions). Portions of antibodies encompasses fragments which include, but are not limited to, Fab, Fab′, F(ab′)₂, Fv, SFv, scFv (single-chain Fv), whether produced by proteolytic cleavage of intact antibodies, such as papain or pepsin cleavage, or produced by recombinant methods, in which the cDNAs for the intact heavy and light chains are manipulated to produce fragments of the heavy and light chains, either separately, or as part of the same polypeptide. In one embodiment of the invention, the antibodies include at least one heavy chain variable region and one light chain variable region, such that the antibody binds a staphylococcal antigen.

MAbs of the present invention encompass antibody sequence corresponding to human and non-human animal antibodies, and hybrids thereof. The term “chimeric antibody,” as used herein, includes antibodies that have variable regions derived from an animal antibody, such as a rat or mouse antibody, fused to another molecule, for example, the constant domains derived from a human antibody. One type of chimeric antibodies, “Humanized antibodies”, have had the variable regions altered (through mutagenesis or CDR grafting) to match (as much as possible) the known sequence of human variable regions. CDR grafting involves grafting the CDRs from an antibody with desired specificity onto the FRs of a human antibody, thereby replacing much of the non-human sequence with human sequence. Humanized antibodies, therefore, more closely match (in amino acid sequence) the sequence of known human antibodies. By humanizing mouse monoclonal antibodies, the severity of the human anti-mouse antibody, or HAMA, response is diminished. The invention further includes fully human antibodies which would avoid, as much a possible, the HAMA response.

Guidance relating to the manipulation of MAb sequences, including the generation of chimeric and humanized antibodies is generally described in Molecular Cloning: A Laboratory Manual, as well as Current Protocols in Molecular Biology (58, 74), Guidance relating more specifically to the manipulation of sequences of the invention may be found in Antibody Engineering, and Antibodies: A Laboratory Manual (75, 76), all of which are incorporated by reference.

The invention includes “modified antibodies,” which as used herein, includes, for example, the proteins or peptides encoded by truncated or modified antibody-encoding genes. Such proteins or peptides may function similarly to the antibodies of the invention. Other modifications, such as the addition of other sequences that may enhance the effector function, which includes the ability to block or alleviate nasal colonization by staphylococci, are also within the present invention. Such modification include, for example, the addition of amino acids to the antibody's amino acid sequence, deletion of amino acids in the antibody's amino acid sequence, substitution of one or more amino acids in the antibody amino acid sequence with alternate amino acids, and isotype or class switching.

In one embodiment, an antibody may be modified in its Fc region to prevent binding to bacterial proteins. The Fc region normally provides binding sites for accessory cells of the immune system. As the antibodies bind to bacteria, and coat them, these accessory cells recognize the coated bacteria and respond to infection. When a bacterial protein binds to the Fc region near the places where accessory cells bind, the normal function of these cells is inhibited. For example, Protein A, a bacterial protein found in the cell membrane of S. aureus, binds to the Fc region of IgG near accessory cell binding sites. In doing so, Protein A inhibits the function of these accessory cells, thus interfering with clearance of the bacterium. To circumvent this interference with the antibacterial immune response, the Fc portion of the antibody of the invention may be modified to prevent nonspecific binding of Protein A while retaining binding to accessory cells.

In light of these various forms of antibodies, the antibodies of the invention will include full length antibodies, fragments thereof, chimeric antibodies, humanized antibodies, human antibodies, and modified antibodies and will be referred to collectively as “MAbs” unless otherwise indicated.

The MAbs of the invention bind to an “antigen” which, as used herein, is a polypeptide sequence, a non-proteinaceous molecule, or any molecule that can be recognized by the immune system. An antigen may be a full-sized staphylococcal protein or molecule, or a fragment thereof, wherein the fragment is either produced from a recombinant cDNA encoding less than the full-length protein or derived from the full-size molecule or protein. Such fragments may be produced via enzymatic processes such as proteolysis or hydrolysis. An antigen may also be a polypeptide sequence that encompasses an epitope of a staphylococcal protein, wherein the epitope may not be contiguous with the linear polypeptide sequence of the protein. The DNA sequence encoding an antigen may be identified, isolated, cloned, and transferred to a prokaryotic or eukaryotic cell for expression by procedures well known in the art (57). An antigen may also be a synthetically produced macromolecule or fragment there-of which elicits an immune response. An antigen may be 100% identical to a region of the staphylococcal protein amino acid sequence, or it may be at least 95% identical, or at least 90% identical, or at least 85% identical. An antigen may also have less identity with the staphylococcal molecule or protein amino acid sequence, provided that it still be able to elicit antibodies the bind to a native staphylococcal molecule or protein. Surface antigens are antigens that are accessible to an antibody when the antigen is in the configuration of the whole intact bacterium, i.e., the antigen is not inside the cell cytoplasm. Virulence antigens (some of which are surface antigens) are antigens that are involved in the pathogenic process, causing disease in a host. Virulence antigens include, for example, LTA, peptidoglycan, toxins, fimbria, flagella, and adherence antigens. Adherence antigens mediate the ability of a staphylococcal bacterium to adhere to the surface of the nares. An antigen may also be a non-proteinaceous component of staphylococci such as a carbohydrate or lipid. For example, peptidoglycan and lipoteichoic acid are two non-proteinaceous antigens found in the cell wall of staphylococci. Antigens may also include fragments of non-proteinaceous molecules as long as they elicit an immune response.

The term “epitope,” as used herein, refers to the region of the staphylococcal antigen that is bound by an antibody. The epitope may be contiguous in the linear polypeptide chain or cell surface macromolecule, or it may encompass. two or more non-adjacent regions of amino acid sequence or fragments of a non-proteinaceous molecule.

An antibody is said to bind, or specifically bind, to an antigen or epitope if the antibody gives a signal by an ELISA assay that is at least two fold, at least three fold, at least five fold, or at least ten fold greater than the background signal.

As used herein, “treatment” encompasses any discernable, medically meaningful, or statistically significant reduction, amelioration, alleviation, or eradication of existing colonization as well as blocking or prophylaxis against future colonization. A “medically meaningful” treatment encompasses any treatment that improves the condition of a patient; improves the prognosis for a patient; reduces morbidity or mortality of a patient; or reduces the incidence of morbidity or rates of mortality from the bacterial infections addressed herein, among a population of patients. The specific determination or identification of a “statistically significant” result will depend on the exact statistical test used. One of ordinary skill in the art can readily recognize a statistically significant result in the context of any statistical test employed, as determined by the parameters of the test itself. Examples of these well-known statistical tests include, but are not limited to, χ² Test (Chi-Squared Test), Students t Test, F Test, M test, Fisher Exact Text, Binomial Exact Test, Poisson Exact Test, one way or two way repeated measures analysis of variance, and calculation of correlation efficient (Pearson and Spearman).

A MAb of the invention is said to “alleviate” staphylococcal nasal colonization if it is able to decrease the number of colonies in the nares of a human or non-human mammal when the MAb is administered before, concurrently with, or after exposure to staphylococci, whether that exposure results from the intentional instillation of staphylococcus or from general exposure. For instance, a MAb is considered to alleviate colonization if the number of bacterial colonies that can be grown from a sample of nasal tissue is decreased after administering the MAb. A MAb alleviates colonization, as in the nasal colonization assays described herein, when it reduces the number of colonies by at least 25%, at least 50%, at least 60%, at least 75%, at least 80%, at least 90%, or by 100%. Another term to describe 100% alleviation would be “eradication.”

A MAb is said to “block” staphylococcal colonization if it is able to prevent the nasal colonization of a mammal when the MAb is administered prior to, or concurrently with, exposure to staphylococci, whether by intentional instillation or otherwise into the nares. A MAb blocks colonization, as in the nasal colonization assay described herein, if no staphylococcal colonies can be grown from a sample of nasal tissue or nasal swab taken from a mammal treated with the MAb of the invention for an extended period such as 12 hours or longer, 18 hours or longer, or 24 hours or longer compared to control mammals.

In a clinical or veterinary setting, the presence or absence of nasal staphylococcal colonization in a patient is determined by culturing nasal swabs on an appropriate bacterial medium and often involves an enrichment step. These cultures are scored for the presence or absence of staphylococcal colonies. In this type of qualitative assay system, it may be difficult to distinguish between blocking and alleviation of staphylococcal colonization. Thus, for the purposes of qualitative assays, such as nasal swabs, a MAb “blocks” colonization if a patient at risk for nasal colonization, who at the time of treatment tests negative for nasal colonization, remains negative for nasal colonization for an extended period, such as 12 hours or longer or 24 hours or longer. A MAb “alleviates” staphylococcal nasal colonization in a patient if it causes a discernable decrease in the frequency of positive cultures taken from the patient or significantly reduces the number of S. aureus recovered by nasal swabbing from a patient who is already positive for staphylococci before the MAbs of the invention are administered.

Because a goal of the invention is to reduce the frequency of S. aureus infections, including nosocomial infections, the instillation of an effective amount includes that sufficient to demonstrate a discernable, medically meaningful, or statistically significant of decrease in the likelihood of staphylococcal infection, for example systemic infection, or infections at the site of trauma or surgery. Such demonstrations may encompass, for example, animal studies or clinical trials of patients at risk, including premature infants, persons undergoing inpatient or outpatient surgery, burn victims, patients receiving indwelling catheters, stents, joint replacements and the like, geriatric patients, and those with genetically, chemically or virally suppressed immune systems.

Thus, the MAbs of the invention are administered to block and/or alleviate staphylococcal nasal colonization. Administration (instillation) of an “effective amount” of the MAb results in a mammal that exhibits any of: 1) no nasal colonization by staphylococci for at least 12 hours after administration, 2) a discernable, medically meaningful, or statistically significant decrease in the number of staphylococcal colonies in the nares, or 3) a discernable, medically meaningful, or statistically significant decrease in the frequency of positive cultures taken from the nares, or 4) a discernable, medically meaningful, or statistically significant decrease in the frequency of staphylococcal infections.

“Instillation” encompasses any delivery system capable of providing a effective amount of a MAb to the mammalian nares. Representative and non-limiting formats include drops, sprays, powders, aerosols, mists, catheters, tubes, syringes, applicators for creams, particulates, pellets, and the like. Also encompassed within the invention are kits comprising a composition containing one or more MAbs of the invention, in connection with an appropriate delivery device or applicator for the composition, for example: catheters, tubes, sprayers, syringes, atomizers, or other applicator for creams, particulates, pellets, powders, liquids, gels and the like.

The invention may be practiced with various nasal delivery vehicles and/or carriers. Such vehicles increase the half-life of the MAbs in the nares following instillation into the nares. These carriers comprise natural polymers, semi-synthetic polymers, synthetic polymers, liposomes, and semi-solid dosage forms (41, 44, 45, 55, 56, 61, 63, 64). Natural polymers include, for example, proteins and polysaccharides. Semi-synthetic polymers are modified natural polymers such as chitosan, which is the deacetylated form of the natural polysaccharide, chitin. Synthetic polymers include, for example, dendrimers, polyphosphoesters, polyethylene glycol, poly (lactic acid), polystyrene sulfonate (PSSA), and poly (lactide coglycolide). Semi-solid dosage forms include, for example, creams, ointments, gels, and lotions. These carriers can also be used to microencapsulate the MAbs or be covalently linked to the MAbs.

In one embodiment, the MAbs of the invention comprise, or are covalently or non-covalently bound to a carrier particle, which may be formulated as a powder, spray, aerosol, cream, gel, etc for application to the nares. In one embodiment, the MAbs are coated onto a carrier particle core in a dissolvable film, which may comprise a mucoadhesive. The carrier particle core may be inert, or dissolvable.

The present invention also discloses a pharmaceutical composition comprising the MAbs together with a pharmaceutically acceptable carrier, which may be, for example, a powder, cream, or liquid. Pharmaceutically acceptable carriers. include sterile liquids, such as water, oils, including petroleum oil, animal oil, vegetable oil, peanut oil, soybean oil, mineral oil, sesame oil, and the like. Suitable pharmaceutical carriers are described in Remington's Pharmaceutical Sciences, 18th Edition (56), incorporated by reference.

In an additional aspect, the MAbs are conjugated to polymers, such as polysaccharides, or any carrier that will covalently link antibodies prior to their administration. This conjugation may serve to increase the antibodies' valency and thereby increase the effectiveness of the antibodies.

Another aspect of the invention is a method of blocking or alleviating secondary staphylococcal infections in patients with respiratory viral infections, transplant patients, HIV infected patients, burn patients, patients with intravascular devices, and other such patients that are subject to secondary infection by administering the MAbs and preparations noted above.

The method of the invention also includes the blocking or alleviation of nasal colonization by any clinical isolate of staphylococci, including any of the various capsule types, as well as strains that are resistant to methicillin, vancomycin, mupirocin and other antibiotics. Furthermore, the invention has the added benefit of inhibiting the spread of antibiotic-resistant strains of staphylococci to the community by blocking nasal colonization in people released from health care settings, a primary reservoir for antibiotic-resistant strains of staphylococci.

Among the staphylococcal antigens against which the MAbs are directed are antigens that play a role in microbial adherence. Microbial adherence to host tissue is a critical early step in colonization by many pathogens. After organisms penetrate the nonspecific mechanical defenses of the host, they bind to various surface receptors of the host using a number of different ligands. Several surface molecules of S. aureus have been identified as potentially playing a role in the initial adherence of the bacteria to cells; these include teichoic acids, lipoteichoic acid (1, 2, 7, 10, 15, 16, 65, 68), Protein A (23), fibronectin binding protein (43, 53), collagen binding protein (23), and the fibrinogen binding protein (27, 40). These adherence factors may mediate attachment of S. aureus to nasal mucosal cells (3, 28, 58), traumatized or disrupted skin (5, 50, 51), and endothelial cells (26, 60), thereby initiating nasal colonization or other infections. Various model systems have been developed to study the binding of these factors to their specific receptors (5, 28, 30, 57). Interference with these factor/receptor interactions often results in blocking of staphylococcal adherence to various tissues. This invention provides MAbs against the staphylococcal antigens that play a role in adherence in the anterior nares.

In addition, the antigens that bind the MAbs of the invention may play a role in virulence. For example, peptidoglycan and LTA can synergize to cause systemic shock. The antigens that bind the MAbs of the invention may also play a role in bacterial survival. For example, alterations in the peptidoglycan molecule can confer antibiotic resistance. Lipoteichoic acid is also involved in recruitment of divalent cations, which also enhance survival. Thus, the MAbs of the invention may decrease virulence and/or the survival of staphylococci in the anterior nares.

Antibodies are very effective in eliminating systemic infections of staphylococci (our data, not shown, and 9, 52, 54). Polyclonal antibody studies have demonstrated that an antibody-based approach may be effective to eliminate adherence of staphylococci to fibrinogen (51 S. aureus adherence to fibronectin was antagonized by anti-staphylococcal immunoglobulin G (IgG)antibodies that were purified from human plasma (67). Blocking of adherence was directly related to the extent of IgG binding to the staphylococcal isolate that was used. More recently, rat antibodies against the staphylococcal fibronectin binding protein (one of the S. aureus adhesions) decreased adherence of radiolabeled S. aureus to immobilized fibronectin (59). Importantly, those antibodies which blocked adherence also maintained their capacity to induce opsonization (59). In another study using primary epithelial cells from bovine mammary glands, serum taken from cows immunized with a whole cell staphylococcal vaccine inhibited the staphylococci binding to epithelial cells (49).

Thus, some polyclonal antibodies extracted from serum or plasma have been shown to block staphylococcal attachment and colonization. However, anti-staphylococcal MAbs that block nasal colonization have not been previously described. MAbs of known specificities avoid problems of varying potency and blood borne pathogens and can be directed against specific staphylococcal targets to decrease the risk of cross-reactivity. In addition, unlike polyclonal antibodies, the MAbs may be modified by standard molecular biology techniques to exhibit varied Fc portions or modified Fc portions. Modifications of this type can be extremely important to the antibody's effectiveness against certain pathogens. For example, part of the mechanism by which S. aureus escapes the humoral immune response is the ability of Protein A to bind the Fc portion of antibodies. This binding interaction decreases the antibody's capacity to mediate clearance of the bacterium.

In addition, the isotype of an IgG antibody can have profound effects on the antibodies' localization in the body and its interaction with the various immuno-regulatory cells of the body such as T cells, dendritic cells, and macrophages. Modified recombinant antibodies have the advantage in that antibodies having different functionalities in the body can be created while maintaining the same binding activity. This modification is accomplished, for example, by fusing the variable regions with alternative IgG constant regions, thus changing the antibody's isotype.

The use of monoclonal anti-staphylococcal antibodies permits the presentation of different specific MAbs that bind different bacterial antigens. Thus, in one aspect, the invention provides a method for alleviating or blocking colonization by S. aureus, ultimately reducing nasal carriage of S. aureus, by instilling one or more of these MAbs directly into mammalian nares. Because of the developing resistance to antibiotics, this approach may prove to be the most effective in both the long and short-term management of staphylococci. The resulting cost savings, from interventions that could reliably inhibit attachment of S. aureus to the nasal mucosa in both the out-patient setting or in a hospital setting, would be significant both by alleviating or blocking hospital acquired infection and by reducing the dissemination of antibiotic-resistant organisms to the community.

As part of this invention, anti-staphylococcal MAbs have been developed and chimerized in our laboratory. Specifically, a chimeric, anti-staphylococcal lipoteichoic acid (“LTA”) monoclonal antibody (A110) has already been evaluated, as shown in Examples 1 and 2, and as set forth in Ser. No. 09/097,055, specifically incorporated by reference. Lipoteichoic acid MAbs were evaluated based on reports (1, 2, 10, 15, 16, 65) suggesting a role of lipoteichoic acid in the initial attachment of staphylococci to epithelial cells. Many investigators have shown that bacterial binding via LTA may be the first step in mediating attachment for many gram positive bacteria to eukaryotic cells (1, 6, 11, 25, 46, 65). Antibodies to LTA can block adherence of staphylococci to fibrin platelet clots (15). Yokoyama et al. (71) suggested that antibodies to S. aureus LTA, present in human serum, may block colonization at the mucosal membrane. Yokoyama's study addressed the role of polyclonal anti-staphylococcal antibodies generated in patients who had been naturally exposed to staphylococci. Yokoyama did not employ MAbs generated as described here nor did Yokoyama disclose methods of using MAbs for intranasal application. With these studies as a background and with the anti-staphylococci MAbs that we have generated, the invention further provides single MAbs or combinations of MAbs that are effective in alleviating or blocking colonization of S. aureus in the anterior nares.

In one aspect, the MAbs of the invention are instilled into the nares of humans. Intranasal administration of antibodies has been reported in the literature as effective in treating a number of conditions. In comparing the efficacy of IgA versus IgG MAbs, Mazanec et al. demonstrated that intranasal application of anti-Sendai virus antibodies afforded significant protection to intranasal challenge with the virus and the efficacy of the two isotypes were equivalent (39). Local application of an anti-Streptococcus mutans specific monoclonal antibody to the teeth of human volunteers prevented recolonization by indigenous S. mutans (36). This protection was seen as late as three days after application of the antibody. In a different model of bacterial infection, intranasal administration of intravenous immunoglobulin (IVIG) exerted significant anti-staphylococcal activity in a mouse model of pneumonia (54). In this study, polyclonal IVIG was introduced in greater volumes to ensure delivery into the lungs via the nose in order to inhibit bacterial growth in the lungs. Thus, none of these studies recognized the benefits of MAb administration to nasal mucosa for blocking or alleviating bacterial nasal colonization.

In addition, the MAbs of the invention work independently of the normal supportive mechanisms in the immune response that enhance antibody activity against a pathogen. An example of such a supportive mechanism is the complement cascade. When a MAb is introduced into a host systemically, the MAb will circulate and eventually specifically bind an antigen. When this occurs, the MAb/antigen complex then triggers activation of the complement pathway. Ultimately, proteins generated by activation of the complement cascade will bind to MAbs that are in turn bound to a specific antigen on the surface of a pathogen, such as a bacterium. When these complement proteins bind MAbs, the bacterium is marked for destruction by a phagocytic cell. In contrast, the MAbs of the invention are administered to the nares. In this location of the host, the MAb does not have access to the complement cascade. Rather, the ability to block and alleviate staphylococcal colonization directly, without the aid of any supportive mechanism, is a unique property of the MAb of the invention.

The MAbs of the invention may be administered in conjunction with other antibiotic anti-staphylococcal drugs including antibiotics like mupirocin and bacitracin; anti-staphylococcal agents like lysostaphin, lysozyme, mutanolysin, and cellozyl muramidase; anti-bacterial peptides like nisin; and otherlantibiotics, or any other lanthione-containing molecule, such as nisin or subtilin.

In view of the disclosure provided, the administration of the MAbs of the invention is within the know-how and experience of one of skill in the art. In particular, the amount of MAbs required, combinations with appropriate carriers, the dosage schedule and amount may be varied within a wide range based on standard knowledge in the field without departing from the claimed invention. For example, doses may range from 1 to 4 times daily giving 0.1 to 20 mg per dose. Specifically, in a typical dosing schedule, the amount of MAb administered would be 2-4 times per day at 0.1-3 mg per dose, a dose known to be effective with an inoculum of 10⁸ S. aureus bacteria, an amount of bacteria known to ensure 100% colonization in an animal model (30). Such a dosing regimen would be effective on patients either admitted to the hospital for surgical procedures, patients suffering from various conditions that predispose them to staphylococcal infections, convalescing patients, infants with immature immune systems, or prior to a patients' release from hospitals. A patient can be any human or non-human mammal in need of prophylaxis or other treatment. Representative patients include any mammal subject to S. aureus or other staphylococcal infection or carriage, including humans and non-human animals such as mice, rats, rabbits, dogs, cats, pigs, sheep, goats, horses, primates, ruminants including beef and milk cattle, buffalo, camels, as well as fur-bearing animals, herd animals, laboratory, zoo, and farm animals, kenneled and stabled animals, domestic pets, and veterinary animals.

The present invention is further illustrated by the following examples that teach those of ordinary skill in the art how to practice the invention. The following examples are merely illustrative of the invention and disclose various beneficial properties of certain embodiments of the invention. The following examples should not be construed as limiting the invention as claimed.

### EXAMPLES

Table 1 lists the MAbs in our laboratory to date. These MAbs are directed against antigens on staphylococci. More specifically, these MAbs are directed against surface antigens.

TABLE 1
MAbs

| Monoclonal Antibody | Target | Block Colonization |
|---|---|---|
| A110¹ | Lipoteichoic acid (LTA) | Yes |
| A110 Fc | Lipoteichoic acid (LTA) | Yes |
| A120² | Lipoteichoic acid (LTA) | n.t.³ |
| 99-110FC12 IE4⁴ | Peptidoglycan⁸ | n.t. |
| MAb-11-232.3⁵ | Peptidoglycan⁸ | Yes |
| MAb-11-248.2⁶ | Peptidoglycan⁸ | n.t. |
| MAb-11-569.3⁷ | Peptidoglycan⁸ | n.t. |

¹See U.S. application Ser. No. 09/097,055, and related application titled Multifunctional Monoclonal Antibodies Directed to Peptidoglycan of Gram-positive Bacteria, filed concurrently herewith, both of which are expressly incorporated by reference.
²A120 is a chimeric MAb disclosed in a related application titled Opsonic Monoclonal and Chimeric Antibodies Specific for Lipoteichoic Acid of Gram Positive Bacteria filed concurrently herewith and expressly incorporated by reference.
³n.t. = not yet tested
⁴Deposited in the ATCC Sep. 21, 2000 under Accession Number PTA-2492.
⁵Also known as QED 15702 (Biosciences, Inc.)
⁶Also known as QED 15703 (Biosciences, Inc.)
⁷Also known as QED 15704 (Biosciences, Inc.)
⁸These MAbs are also disclosed in a related application titled Multifunctional Monoclonal Antibodies Directed to Peptidoglycan of Gram-positive Bacteria filed concurrently herewith and expressly incorporated by reference.

Other MAbs are likewise encompassed by the invention, particularly those MAbs directed against other epitopes implicated in the adherence, survival or virulence of staphylococcal bacteria.

### Example 1

### MAb A110 Binds with Whole S. aureus and S. epidermidis

Thus far, the anti-staphylococcal LTA monoclonal antibody (A110) has been developed, chimerized, and tested as set forth in U.S. Ser. No. 09/097,055, filed Jun. 15, 1998, incorporated herein by reference. This MAb is currently being manufactured under GMP conditions in preparation for clinical trials. We tested the reactivity of the MAb and found that it binds with whole S. aureus types 5 (SA5) and 8 (SA8), as well as several types of Staphylococcus epidermidis including the highly virulent type 2 isolate Hay as shown in Table 2.

The data in Table 2 was generated using A110 that was purified with a protein G column (Pharmacia). The whole cell ELISA assay was performed to measure the ability of MAbs to bind to live bacteria. Various types of bacteria may be used in this assay, including S. aureus type 5, type 5-USU, type 8, S. epidermidis strain Hay, and S. hemolyticus. Bacteria from an overnight plate culture was transferred to 35 mls of Tryptic Soy Broth (TSB) and grown with gentle shaking for 1.5-2.0 hours at 37° C. The bacteria were then pelleted by centrifugation at 1800-0.2000×g for 15 minutes at room temperature. The supernatant was removed and the bacteria were resuspended in 35-45 mls of phosphate buffered saline (PBS) containing 0.1% bovine serum albumin (PBS/BSA). The bacteria were again pelleted by centrifugation, the supernatant discarded and the bacteria resuspended in PBS/BSA to a percent transmittance (%T) of 65%-70% at 650 nm. From this suspension the bacteria were further diluted 15-fold in sterile 0.9% sodium chloride (Sigma cat. no. S8776, or equivalent), and 100 μl of this suspension was added to replicate wells of a flat-bottomed, sterile 96-well plate.

Each MAb to be tested was diluted to the desired concentration in PBS/BSA containing 0.05% Tween-20 and horseradish peroxidase-conjugated Protein A (Protein A-HRP, Zymed Laboratories) at a 1:10000 dilution (PBS/BSA/Tween/Prot A-HRP). The Protein A-HRP was allowed to bind to the MAbs for 30-60 minutes at room temperature before use, thereby generating a MAb-Protein A-HRP complex to minimize the potential nonspecific binding of the MAbs to the Protein A found on the surface of S. aureus. Generally, several dilutions of test MAb were used in each assay. From each MAb dilution, 50 μl of the MAb-Protein A-HRP complex was added to replicate wells and the mixture of bacteria and MAb-Protein A-HRP complex was incubated at 37° C. for 30-60 minutes with gentle rotation (50-75 rpm) on an orbital shaker.

Following the incubation, the bacteria were pelleted in the plate by centrifugation at 1800-2000×g. The supernatant was carefully removed from the wells and 200 μl of PBS. BSA containing 0.05% Tween-20 (PBS/BSA/Tween) was added to all wells to dilute unbound reagents. The bacteria were again pelleted by centrifugation.and the supernatant was removed. One hundred microliters of TMB substrate (BioFx, Inc. cat. no. TMBW-0100-01) was added to each well and the reactions were allowed to proceed for 15 minutes at room temperature. The reactions were stopped by adding 100 μl of TMB stop reagent (450 nm Stop Reagent; BioFx, Inc. catalog no. STPR-0100-01). The absorbance of each well was determined using a microplate reader fitted with a 450 nm filter. In this assay, the intensity of the color development was directly proportional to the binding of the MAbs to the bacteria. Control wells contained bacteria and Protein A-HRP without MAb.

Using this modified whole cell ELISA protocol, peroxidase labeled Protein A was mixed with the purified Al 10 and then reacted with S. aureus type 5 (SA5) and S. aureus type 8 (SA8) obtained from ATCC at Accession Nos. 12602 and 12605, respectively. Both S. aureus serotypes bound to the A110 MAb. This finding was also important since S. aureus serotypes 5 and 8 are commonly associated with human infections. Using this protein A assay, MAb to type 14 pneumococcus did not demonstrate binding to S. epidermidis or S. aureus type 5.

TABLE 2
Immunoassay¹ of Protein G-Purified A110 on S. aureus and S. epidermidis

| | | | Absorbance on Wells Coated with: | | |
|---|---|---|---|---|---|
| | | | S. epidermidis | S. aureus | S. aureus |
| Antibody | Concentration | | (HAY) | Type 5 | Type 8 |
| Buffer | | | 0.152 | 0.102 | 0.113 |
| A110 | 3.3 | μg/ml | 3.017 | 1.329 | 3.345 |
| A110 | 1.6 | μg/ml | 2.266 | 1.275 | 2.141 |
| A110 | 0.8 | μg/ml | 1.487 | 0.873 | 1.016 |
| A110 | 0.4 | μg/ml | 0.951 | 0.333 | 0.491 |
| Anti-Pn14² | 0.5 | μg/ml | 0.112 | 0.105 | N.D. |

| | | | | |
|---|---|---|---|---|
| NMS² | 1:1000 | 0.101 | 0.090 | 0.082 |

¹A modified ELISA measuring binding of MAb to live organisms. Since Protein A on the surface of S. aureus can bind MAbs through the Fc portion, our immunoassay was modified to avoid non-specific binding of A110 to surface Protein A.
²Normal mouse serum (NMS) and a monoclonal antibody reactive with the polysaccharide from Streptococcus pneumonia type 14 (Pn14) were not reactive and therefore were used as negative controls.

### Example 2

### MAb A110 Binds to LTA Isolated from Several Gram Positive Organisms

A110 also binds lipoteichoic acid isolated from a number of gram positive organisms. Table 3 shows the data from an ELISA, modified as per above, in which the plate wells were coated, using standard techniques, with LTA isolated from different gram positive bacteria including S. aureus, S. mutants, S. pyogenes, and B. subtillus. A goat anti-human heavy chain and light chain antibody conjugated to HRP was used as a secondary antibody (Zymed Inc.). Clearly, A110 bound to LTAs from all bacteria tested.

TABLE 3
Immunoassay of Purified A110 on LTAs from Different Bacteria

| | | Absorbance on Wells Coated With: | | | |
|---|---|---|---|---|---|
| | | S. | S. | S. | |
| Antibody | Concentration | aureus | mutants | pyogenes | B. subtillus |
| Buffer | — | 0.057 | 0.055 | 0.055 | 0.063 |
| A110 | 3 μg/ml | 3.343 | 3.082 | 3.234 | 2.928 |
| A110 | 1 μg/ml | 3.482 | 3.267 | 3.590 | 2.918 |
| A110 | 0.33 μg/ml | 3.084 | 2.817 | 3.016 | 2.622 |
| A110 | 0.11 μg/ml | 2.649 | 2.421 | 2.674 | 2.054 |
| A110 | 0.037 μg/ml | 1.907 | 1.673 | 1.930 | 1.324 |

Examples 3-5 below evaluate the capacity of MAbs to block colonization of mouse anterior nares by S. aureus. Example 6 demonstrates the effect on colony clearance of adding dextran as a conjugate. As Examples 3-5 involved premixture of bacteria with MAb before application, Example 7 below shows that the disclosed anti-LTA and anti-peptidoglycan MAbs are also effective when MAbs are first introduced into the anterior nares followed by bacteria. Example 8 demonstrates that nasally applied MAbs can alleviate colonization even when the colonization was established before antibody application. Example 9 demonstrates the effect of different carrier substances on the retention of MAbs in the whole mouse nose. Example 10 demonstrates that MAbs in PSSA can alleviate established staphylococcal colonization in a single dose.

### Example 3

### Pre-Incubation of MAb A110 with S. aureus Blocks Nasal Colonization

Kiser et al. developed a staphylococcal nasal colonization model in mice to study staphylococcal factors that influence nasal colonization (30). Using this model, we demonstrated that intranasal instillation of A110 in saline (PBS) blocked and/or alleviated S. aureus nasal colonization. Briefly, streptomycin resistant S. aureus type 5 was grown on high salt Columbia agar to promote capsule formation. The bacteria were washed with sterile saline to remove media components and resuspended at ˜10⁸ organisms/animal dose in saline containing various concentrations and combinations of anti-staphylococcal or irrelevant control MAbs. Following a preincubation of 1 hr, the bacteria were repelleted and resuspended in a final volume of 10 μl per animal dose in either saline or saline containing antibody. Mice that have been maintained on streptomycin-containing water for 24 hrs were sedated with anesthesia. Staphylococci were instilled in the nares of the mice by pipetting without contact with the nose.

Generally, following four to seven days during which the animals were maintained on streptomycin-containing water, the animals were sacrificed and the noses removed surgically and dissected. Nasal tissue was vortexed vigorously in saline plus 0.5% Tween-20 to release adherent bacteria, and the saline was plated on Columbia blood agar and tryptic soy agar containing streptomycin to determine colonization.

According to this procedure, streptomycin-resistant S. aureus type 5 (SA5, 1 to 3×10⁸/mouse) was preincubated for 1 hr in saline or saline containing A110 (2-3 mg purified IgG/mouse dose of 1-3×10⁸ bacteria). Following preincubation, the bacteria were pelleted and resuspended in saline or in saline containing A110 (10 μl/mouse dose). Ten mice each were intranasally instilled with SA5 in saline or SA5 in A110. Table 4 tabulates three experiments showing that nasal application of A110 in PBS blocks and alleviates staphylococcal nasal colonization.

TABLE 4

| | |
|---|---|
| Number of | Average number of |
| mice colonized | colonies recovered |

Experiment 1: nasal tissue harvested at seven days
1 × 10⁸ SA5 instilled in:

| | | |
|---|---|---|
| Sterile Saline | 7/8 | 35 |
| A110 (3 mg/mouse dose) | 3/8 | 10 |

Experiment 2: nasal tissue harvested at four days
1 × 10⁸ SA5 instilled in:

| | | |
|---|---|---|
| Sterile Saline | 11/11 | 30 |
| A110 (2 mg/mouse dose) | 6/11 | 10 |

Experiment 3: nasal tissue harvested at seven days
3 × 10⁸ SA5 instilled in:

| | | |
|---|---|---|
| Sterile Saline | 10/10 | 19 |
| A110 (2 mg/mouse dose) | 5/10 | 8 |

### Example 4

### Blocking of Nasal Colonization is Specific to the Presence of Anti-Staphylococcal Antibodies

To ensure that the blocking of nasal colonization obtained with our MAbs was specific for anti-staphylococcal antibodies, we examined the capacity of an irrelevant control chimerized IgG to block staphylococcal nasal colonization. The control was Medi 493, a chimeric IgG1 MAb against RSV (29, MedImmune). In the same experiment, we also tested MAb-11-232.3, a MAb specific for a staphylococcal S. aureus surface antigen, for its capacity to block colonization. MAb-11-232.3 (QED Biosciences) was produced by immunizing mice with UV-inactivated whole S. aureus, and the MAb was subsequently shown to bind to peptidoglycan. This experiment was conducted as described above and the results are presented in Table 5, which shows that MAb-11-232.3 in saline blocked and alleviated staphylococcal nasal colonization in mice but that an anti-RSV MAb in saline had no effect.

TABLE 5

| | | Average number of |
|---|---|---|
| | Number of mice | colonies recovered per |
| 2 × 10⁸ SA5 instilled with: | colonized | mouse |
| Sterile Saline | 9/9 | 70 |
| MAb-11-232.3 (2 mg/mouse | 3/8 | 8 |
| dose) | | |
| Medi 493 (2 mg/mouse | 9/9 | 137 |
| dose) | | |

Table 5 shows that both the number of mice colonized and the number of colonies recovered per colonized mouse were decreased in an antibody-specific manner by the anti-S. aureus surface antigen MAb. All of the mice in the saline control and the irrelevant chimerized IgG control groups were colonized with S. aureus, but only three out of eight mice were colonized in the MAb-11-232.3 group. The number of colonies recovered per mouse in the MAb-11-232.3 group was decreased as compared with the other two groups. Therefore, the effect was specific for anti-staphylococcal surface antigen MAbs and was not just a general consequence of antibody binding to surface Protein A on the staphylococci. Additional MAbs against S. aureus peptidoglycan were generated, MAb-11-248.2 and MAb-11-569.3 (QED Biosciences), which should demonstrate similar inhibitory effects on S. aureus colonization as described above. Studies are in progress to affirm the effectiveness of MAb-11-248.2 and MAb-11-569.3 in the in vivo mouse model described above.

### Example 5

### Fc Mutant MAb that Bind Staphylococci also Block Nasal Colonization

We also have developed a form of A110 in which the Fc region has been modified to inhibit the normal binding of the Fc domain to staphylococcus Protein A. To generate the Fc mutant antibody, we mutated the CH3 domain of IgG1 that normally binds Protein A. Specifically, we employed a method of mutagenesis (47, 8) based on the use of two complementary oligonucleotides containing the mutations desired and the restriction endonuclease DpnI to digest the parental (non-mutated) DNA strands following the protocol provided by Stratagene, Inc. The sequences of the two oligonucleotides used for the mutagenesis process are:

| | | |
|---|---|---|
| IgG1Fc3S: | 5′-GCTCTGCACAACCGCTTCACGCAGAAGAGCC-3′ and | (SEQ ID NO:3) |
| IgG1Fc3AS: | 5′-GGCTCTTCTGCGTGAAGCGGTTGTGCAGAGC-3′. | (SEQ ID NO:4) |

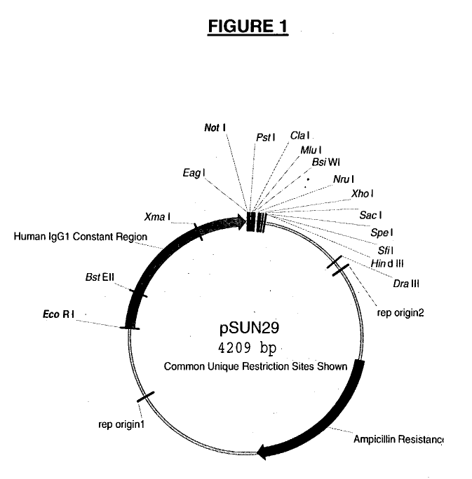

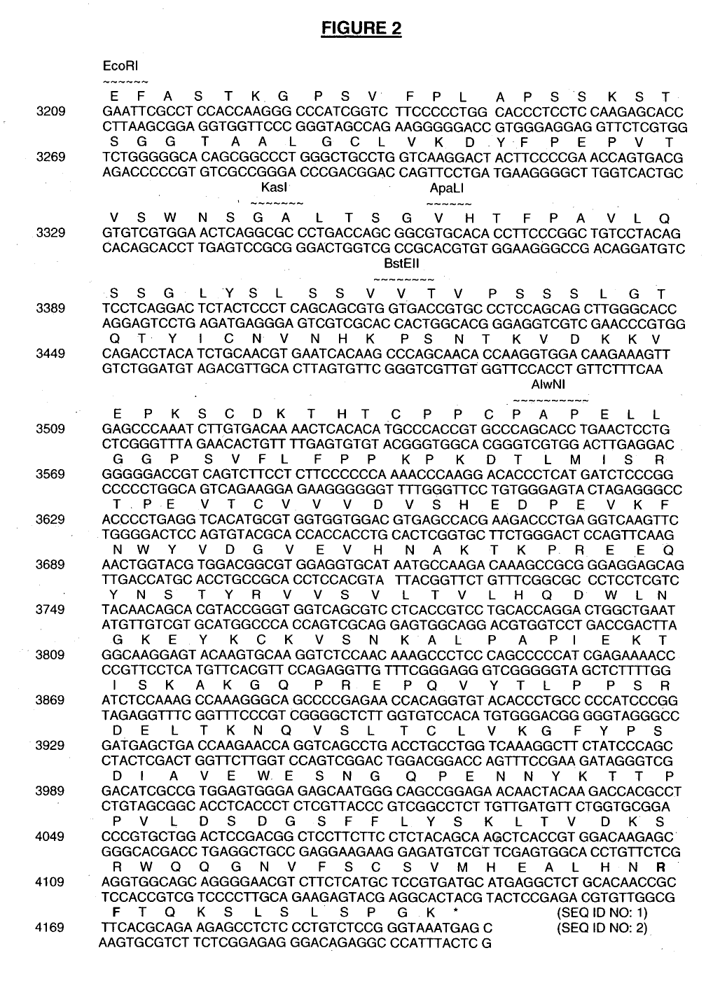

The plasmid pSUN29, the pSL1180 plasmid (Pharmacia) containing human IgG1 coding region, was used as a template for the mutagenesis process (FIG. 1). The IgG1Fc3S and IgG1Fc3AS oligonucleotides were combined with pSUN29, dNTPs, reaction buffer, and PfuTurbo DNA polymerase. The reaction was carried out as described in the Quickchange Mutagenesis System (Stratagene). Following the DpnI digestion the sample was diluted 1:10 in water, and 2 μL was used to transform Ultracompetent XL2 Blue cells (Stratagene) per the manufacturer's procedure. Plasmid clones containing DNA inserts were identified using diagnostic restriction enzyme digestion using EcoRI and NotI following plasmid DNA purification (Qiagen) from overnight cultures of well-isolated individual bacterial colonies. The DNA sequence of plasmids containing inserts of the appropriate size (˜1000 bp) was then confirmed to contain the desired mutations, H435R and Y436F. These amino acids match those found at the homologous location in the human IgG3 isotype. The final consensus DNA and amino acid sequence of the heavy chain constant region is shown in FIG. 2.

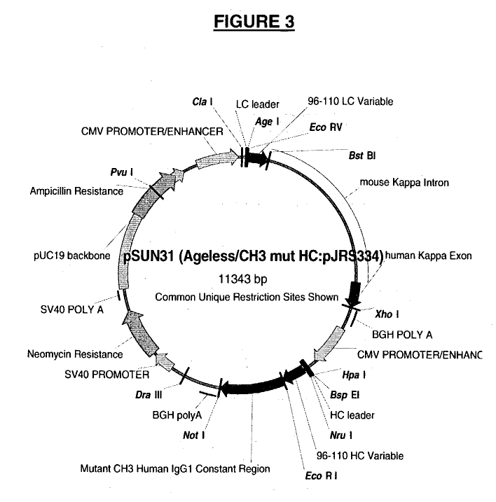

The mutated IgG1 constant region was combined with the A110 variable region to make MAb A110 Fc. Specifically, the plasmid pSUN30 was digested with the restriction endonucleases EcoRI and NotI (New England Biolabs), and the DNA fragment containing the mutated human IgG1 coding sequence was gel purified using the Qiaquick spin column DNA/Gel isolation system (Qiagen). The plasmid pJRS334 is a mammalian expression plasmid that contains a cDNA sequence encoding MAb A110. Plasmid pJRS334 was digested with EcoRI and NotI and the vector backbone fragment was gel purified using the Qiaquick spin column system described above. The pJRS334 plasmid backbone, and the IgG1 mutant insert were ligated per manufacturer's instructions (New England Biolabs), and the ligation products were transformed into XL2blue cells (Stratagene). Plasmid clones were purified form overnight cultures of individual bacterial colonies using the Qiaprep system (Qiagen). The DNA sequence of plasmids containing inserts of the appropriate size (1000 bp) was then determined by fluorescence-labeled DNA sequencing using an ABI Sequencer. Plasmid pSUN31 contained the Fc mutant of the A110 antibody, A110 Fc, of the correct size and sequence. FIG. 3 shows a schematic plasmid map of pSUN31.

An antibody production ELISA was used to determine whether COS cells transfected with pSUN31 produce the A110 Fc antibody. Briefly, in this assay, an anti-human IgG antibody is bound to the wells of a 96-well plate. Supernatants from the transfected COS cells are added to the wells, followed by an anti-human kappa HRP-conjugated antibody. The presence of the HRP moiety is detected using TMB substrate (Kirkgaard & Perry Laboratories), which, following incubation in the presence of HRP, has a measurable absorbance at 450 nm. Therefore, the wells of the 96-well plate will have an absorbance at 450 nm above background only if the supernatant produces an antibody that contains both a human IgG domain and a human kappa domain.

One microgram of plasmid pSUN31 was transfected into COS cells using Superfect (Qiagen) in 6 well tissue culture cells as described by the manufacturer. After three days the supernatant was assayed in an antibody production ELISA. Antibody production ELISA assays were performed in 8-well strips from 96-well microtiter plates (Maxisorp F8; Nunc, Inc.), coated with a 1:500 dilution of Goat anti-Human IgG antibody (Pierce) in PBS. The plates were covered with film (“pressure sensitive film” Falcon, Becton Dickinson) and incubated overnight at 4° C.

Plates were washed once with Wash solution (Imidazole/NaCl/0.4% Tween-20), and 100 μL of culture supernatant was added to duplicate wells and allowed to incubate for 30 minutes on a plate rotator at room temperature. The plates were washed five times with Wash solution. 100 μl of goat anti-human kappa-HRP conjugate (Southern Biotechnologies), diluted 1:800 in sample diluent (10% FBS in PBS) was added to the samples, and the plates were incubated on a plate rotator for 30 minutes at room temperature. The samples were washed five times with Wash solution, and 100 μL of TMB developing substrate (Kirkgaard & Perry Laboratories) was added per well, and the plates were incubated on a plate rotator at room temperature for 5 minutes. The reactions were stopped with 100 μL of Quench buffer (Kirkgaard & Perry Laboratories) and the absorbance of each well at 450 nm was determined using an automated microtiter plate ELISA reader (Ceres UV900HI, Bio-tek, Winooski, Vt.).

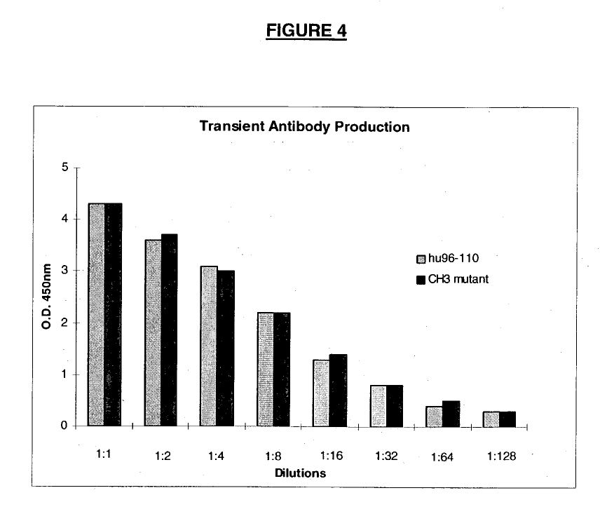

As a positive control, the parent antibody, A110, was included in the assay. This assay demonstrates that transfection of COS cells with pSUN31 results in production of an antibody that contains both a human IgG domain and a human Kappa domain (see FIG. 4).

The supernatants were then assayed for the ability of the expressed antibodies to bind to S. aureus lipoteichoic acid (LTA) and S. aureus Protein A (SpA). Relative to the parent antibody, A110, the Fc mutant antibody, A110 Fc, is expected to no longer bind to protein A, while retaining the ability to bind LTA.

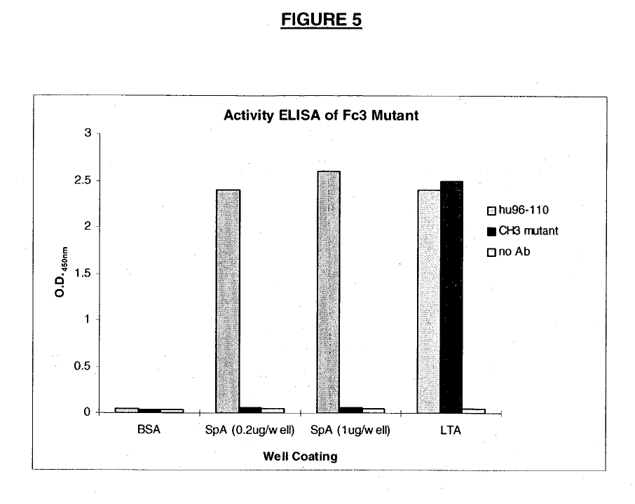

The activity assays were performed in 8-well strips from 96-well microtiter plates (Maxisorp F8; Nunc, Inc.), which had been coated with 1.0 μg S. aureus LTA (Sigma), 0.2 μg SpA (Sigma), or 0.1 μg SpA (Sigma) in 100 μl of PBS. As a negative control, wells were coated with 1.0 μg BSA. The plates were covered with pressure sensitive film and incubated overnight at 4° C. Plates were washed once with Wash solution (Imidazole/NaCl/0.4% Tween-20), and 100 μl of culture supernatant was added to duplicate wells and allowed to incubate for 30 minutes on a plate rotator at room temperature. The plates were washed five times with Wash solution. 100 μl of goat anti-human kappa-HRP conjugate (Southern Biotechnologies), diluted 1:800 in sample diluent (10% FBS in PBS), was added to each well, and then incubated on a plate rotator for 30 minutes at room temperature. The samples were washed five times with Wash solution and then 100 μL of TMB developing substrate (Kirkgaard & Perry Laboratories) was added per well, and the plates were incubated for 5 minutes on a plate rotator at room temperature. The reaction was stopped with 100 μL/well of Quench buffer (Kirkgaard & Perry Laboratories) and the absorbance value of each well at 450 nm was determined using an automated microtiter plate ELISA reader (Ceres UV900HI, Bio-tek, Winooski, Vt.). As a positive control, supernatant from mammalian cells transfected with pJRS334, a plasmid that encodes the parent antibody A110, was used. This assay demonstrates that the transfection of COS cells with pSUN31 results in production of a recombinant antibody that retains the ability to bind to S. aureus LTA but no longer binds to S. aureus Protein A (FIG. 5). This assay also confirms that the parent antibody, A110, binds protein A, while the mutant antibody that has two amino acid changes in its Fc region, A110 Fc, does not.

A cell line stably transfected with the pSUN31 plasmid was generated. Specifically, CHO cells were transfected by electroporation with pSUN31 plasmid that had been linearized by digestion with PvuI restriction endonuclease (New England Biolabs). Briefly, 25 μg of digested pSUN31 plasmid DNA was mixed with 1×10⁷ CHO cells in a total volume of 800 μL of PBS in a 0.4 cm cuvette, and subjected to a pulse of 250 mA, 960 μF. The cells were diluted into 100 ml non-selective media MDM, 10% serum (Hyclone) and 100 μl were added to each well of 10, 96-well microtiter plates. After 24 hours, the media was removed from the 96 well plates and replaced with selective media, MDM, 10% serum containing 750 ug/ml G418. After colonies appeared, the supernatants were assayed for the production of antibody by checking for the inability to bind to S. aureus Protein A and the continued ability to bind to S. aureus LTA.

Antibody production and activity assays for the stable transfectants were performed as described above. These assays demonstrate that the transfection of cells with this plasmid construct can result in the production of a stable cell line that produces a humanized, chimeric, Fc mutant antibody, A110 Fc, that retains the ability to bind to LTA, but no longer binds to Protein A.

The results from testing MAb A110 Fc in PBS in the above described nasal colonization model are presented in Table 6.

TABLE 6

| | | Average number of |
|---|---|---|
| | Number of mice | colonies recovered per |
| 3 × 10⁸ SA5 instilled in: | colonized | mouse |
| Sterile Saline | 10/11 | 240 |
| A110 Fc | 11/11 | 16¹ |

¹One abnormally highly colonized animal from this group was eliminated by Student's T Test.

Even though all animals in the A110 Fc treated group were colonized by S. aureus, the A110 Fc antibody alleviated nasal colonization by S. aureus when antibody was administered with the bacteria. Specifically in the antibody-treated mice, the average number of colonies per nose was greatly decreased as compared to saline treated mice.

### Example 6

### Conjugation of MAb A110 Increases its Effectiveness

We have conjugated the MAbs to various carrier substances in order to increase the number of antigen binding sites on each antibody/carrier conjugate (i.e., the valency). We did this antibody conjugation for the known antibodies and any discovered MAbs capable of blocking colonization of staphylococcal nasal colonization. This conjugation procedure was performed as described in Lees et al. (35). Briefly, antibodies were conjugated to amino ethyl carbamyl dextran (AECM dextran) using heteroligation techniques as follows. Antibodies were acetylthiolated using N-hydroxycuccinimidyl S-acetylthoacetate (SATA, purchased from Bioaffinity Systems, Roscoe, Ill.) and the AECM dextran iodoacetylated using a large excess of N-hydroxycuccinimidyl iodoacetate reagent (Bioaffinity Systems). Antibodies were reacted with 4-8 fold molar excess of SATA for 1-2 hours. Labeling of both the AECM dextran and the antibody was performed in 0.15M HEPES, 2 mMEDTA, pH 7.3. Labeled antibodies and dextran were desalted and mixed at molar ratios of 30-60:1. The pH was raised to 7.5, made 25 mM in hydroxylamine and the reaction allowed to proceed overnight. Unconjugated antibody was removed by gel filtration chromatography on an S400HR (Pharmacia) column. Protein concentration of the conjugate was measured by determination of the optical density at 280 nM using 1.4 OD/mg/ml. The dextran concentration was determined using the method of Monsigny et al 1988. Conjugates were sterile filtered using a 0.2μ Millex GV device (Millipore).

Preliminary experiments have shown that A110 conjugated to high molecular weight dextran induced significantly greater agglutination of S. aureus as compared to unconjugated A110. We have conjugated A110 to dextran through the procedure described above and tested this conjugated MAb in our nasal colonization assay as shown in Table 7 below.

TABLE 7

| | | Average number of |
|---|---|---|
| | Number of mice | colonies recovered per |
| 2 × 10⁸ SA5 instilled with: | colonized | mouse |
| Sterile Saline | 8/8 | 42 |
| 0.5 mg A110 | 7/8 | 13 |
| 0.5 mg A110 dextran | 5/8 | 3 |

Conjugation of A110 to dextran increased the capacity of the antibody to block and/or alleviate nasal colonization. Specifically, the number of mice colonized dropped from 8/8 (control) to 5/8 in the dextran conjugated sample. Further, for those 5 mice still positive, the average number of colonies recovered dropped several fold.

### Example 7

### Pre-Instillation of MAb into the Nares Blocks Nasal Colonization

In all of the above described examples, the MAb in PBS was preincubated with the S. aureus prior to nasal instillation. Realizing that this may not fully mimic the clinical setting, we examined whether MAb could be pre-instilled in mouse nares and then followed by bacterial instillation and still block colonization. In two experiments (summarized in Tables 8 and 9) mice were anaesthetized and A110 in PBS was instilled in the nares. Ten minutes later, S. aureus was instilled in the nares. Following five days, the mice were sacrificed and the noses were plated to detect the presence of S. aureus.

TABLE 8

| | | Average number of |
|---|---|---|
| 2 × 10⁸ SA5 instilled | Number of mice | colonies recovered per |
| following pre-instillation of: | colonized | mouse |
| Sterile Saline | 10/10 | 23 |
| A110 (0.1 mg/animal) | 7/11 | 9 |

TABLE 9

| | | Average number of |
|---|---|---|
| 3 × 10⁸ SA5 instilled | Number of mice | colonies recovered per |
| following pre-instillation of: | colonized | mouse |
| Sterile Saline | 11/11 | 69 |
| A110 (0.1 mg/animal) | 8/11 | 13 |

We also examined the pre-instillation of one of our anti-peptidoglycan MAbs (MAb-11-232.3) in the same manner as described above. The results of this experiment are presented in Table 10.

TABLE 10

| | | Average number of |
|---|---|---|
| 4 × 10⁸ SA5 instilled | Number of mice | colonies recovered per |
| following pre-instillation of: | colonized | mouse |
| Sterile Saline | 9/10 | 11 |
| MAb-11-232.3 | 5/11 | 2¹ |
| (0.09 mg/animal) | | |

¹One abnormally high colonized animal from this group was eliminated by Student's T Test.

In a fourth experiment, we tried combining A110 with MAb-11-232.3 in PBS and pre-instilling this in the mouse nares prior to S. aureus instillation as described above.

TABLE 11

| | | Average number of |
|---|---|---|
| 2.5 × 10⁷ SA5 instilled | Number of mice | colonies recovered per |
| following pre-instillation of: | colonized | mouse |
| Sterile Saline | 8/8 | 14 |
| A110 (0.05 mg/animal) | 7/10 | 2 |
| MAb-11-232.3 | 8/10 | 23 |
| (0.05 mg/animal) | | |
| A110 (0.05 mg/animal) | 5/9 | 4 |
| and | | |
| MAb-11-232.3 | | |
| (0.05 mg/animal) | | |

These experiments demonstrate that it is possible to pre-instill MAb in the nares and then block and alleviate subsequent nasal colonization following challenge with a large inoculum of S. aureus. The effectiveness of the inhibition of colonization is lower with pre-instillation than with co-incubation as documented above, but we believe this is due to the extremely short half-life of MAb in buffer instilled in the nares. Indeed, we have determined that by 5 min post instillation, 90% of MAb is no longer detectable in the nares (data not shown). We are actively pursuing research in delivery of MAb in various formulations to the nares with the intent of substantially increasing the half-life of the MAb.

### Example 8

### Nasal Application of Multiple Doses of MAb in Saline Alleviates Pre-Established Nasal Colonization

We also propose using MAbs to help alleviate established S. aureus nasal colonization. Towards this goal, we sought to determine whether nasal instillation of A110 could alleviate established nasal colonization. Mice were instilled with 6×10⁷ S. aureus. One and three days following instillation of bacteria, saline or A110 in saline was instilled in the nares of the colonized mice. On day five, mice were sacrificed and the noses plated for the presence of S. aureus.

TABLE 12

| 4 × 10⁸ SA5 instilled | | Average number of |
|---|---|---|
| followed by instillation | Number of mice | colonies recovered per |
| of: | colonized after treatment | mouse |
| Sterile Saline | 10/10 | 58 |
| A110 (0.1 mg/animal | 4/11 | 14 |
| on days 1 and 3) | | |

This example demonstrates that it was possible to eradicate and alleviate established nasal colonization by instilling MAbs in the nares of colonized individuals.

### Example 9

Addition of Mucoadhesive Polymers to MAbs Improves Retention Time in the Nares

Rapid clearance of nasal mucous is the major technical hurdle in the administration of nasal therapeutics. The clearance time for materials in the human nose is about 12-15 minutes, although clearance occurs more slowly in the anterior third of the nose because mucous flow there depends on traction generated by cilia on epithelial cells that reside more posterior to the anterior nares (4, 33). Therefore, frequent dosing of MAbs may be required to maintain nasal concentrations effective at blocking S. aureus colonization, which may lead to increased therapy costs. Mucoadhesive polymers such as cellulose and polystyrene derivatives, chitosan, cyclodextrins, and poly-L-arginine have been used in various strategies to increase the residence time of nasally administered drugs (41, 45, 63, 64). However, the vast majority of these delivery systems have focused on increasing systemic absorption of the carrier drug and not specifically on increasing residence time and activity in the nasal mucosa. Nasal delivery systems that will significantly increase the residence time of MAbs and preserve their activity are in development, with the goal of requiring only 1 to 4 times daily administration.

Initial studies evaluated the relative efficacy of four purported mucoadhesive polymers: chitosan, hydroxypropyl cellulose, poly-L-arginine, and PSSA. When A110 was mixed with 0.5% (w/v) of these polymers and added drop wise to the noses of mice, an increase in nasal MAb retention compared to saline administration was observed for all except poly-L-lysine. The greatest retention one hour after administration was achieved with the PSSA solution, 78% versus 43% for saline-MAb treated noses. By three hours, PSSA-Ab microspheres continued to show the greatest retention, with 46% remaining, compared to 15% for antibody in saline. The noses were extracted at various times after administration, washed with PBS/Tween-20, and the antibody measured in an LTA binding ELISA assay. Upon examination by light microscopy, it was discovered that the process of mixing the PSSA with A110 formed microparticles with an approximate size range of 10 to 50 μm. The presence of microparticles may be important based on the observation that encapsulated antibodies are removed from the nose more slowly.

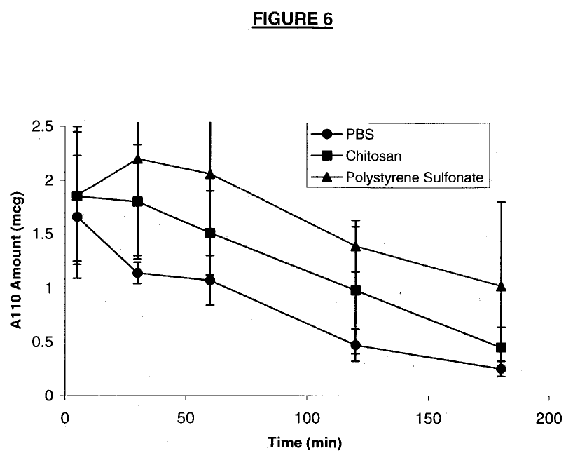

Nasal clearance of A110 mixed with PSSA or chitosan was measured over a three hour period and compared to saline-MAb administration, as shown in FIG. 6. Chitosan (CS) and PSSA prolonged retention of antibody in the mouse noses. The major retention activity of these polymers appears to occur within the first hour after administration, virtually 100% for PSSA and 82% for chitosan compared to 64% for saline. The rate of A110 clearance from the nose appeared to equalize between 1 and 3 hours post-administration for all three vehicles, as indicated by the equal slopes of the chitosan and PSSA sample lines in FIG. 6 between these time points. However, this difference in retention over the first hour after treatment leads to large absolute differences in antibody amounts in the nose at 3 hours, as indicated by the spread between the amount of antibody remaining in chitosan treated mice compared to the amount of antibody remaining in the PSSA treated mice at this time point.

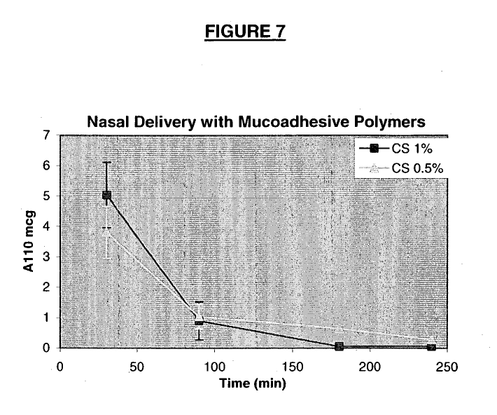

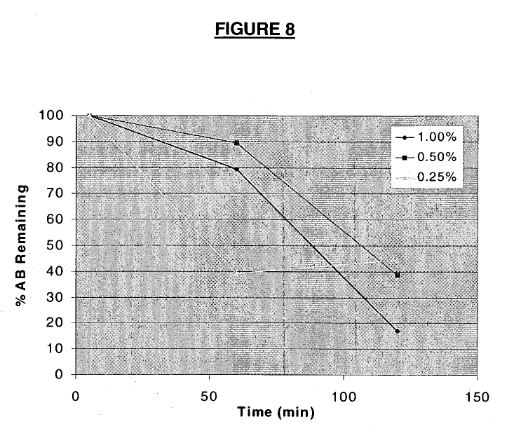

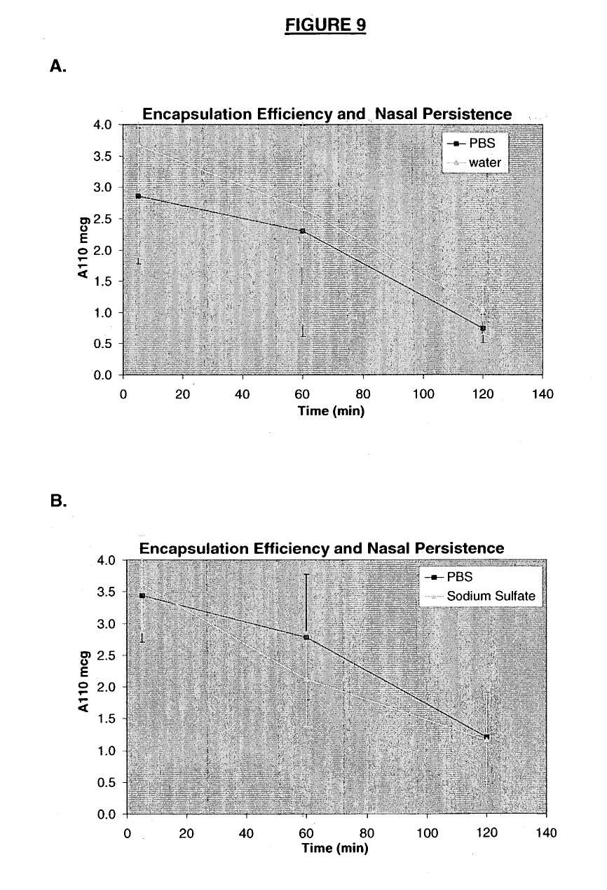

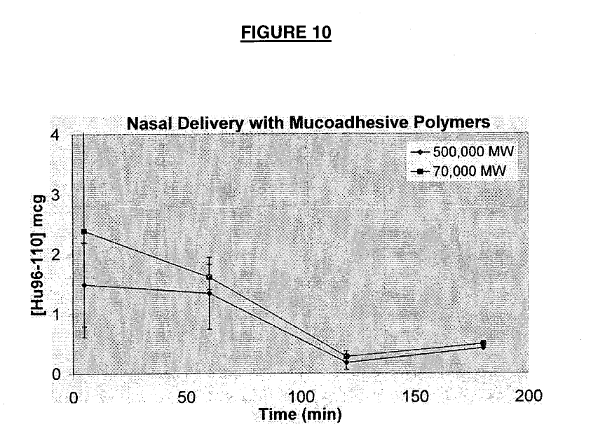

Many variables have been examined in the formulation of antibody with PSSA and CS including: polymer molecular weight, polymer concentration, microsphere size, and salt concentration. FIG. 7 demonstrates that increasing the CS concentration did not increase the retention of antibody in the nose. FIG. 8 shows the effect of polystyrene-Ab microsphere size on antibody retention in the nose. The labels in this figure show the percent of PSSA used in microsphere synthesis and correlates with microsphere size. Microsphere size was estimated to be 0.25% as 50 μm, 0.5% as 25 μm, and 1.0% as 0.5 to 1.0 μm. As indicated in this figure, the microsphere size did not alter the retention of antibody in the nose, suggesting that microspheres of wide ranging size may be effective. FIGS. 9A and 9B demonstrate that salt concentration and type affected the encapsulation efficiency of Ab in the microspheres. In these figures, water, PBS, or sodium sulfate were compared against each other. Water had the lowest encapsulation efficiency and sodium sulfate the highest encapsulation efficiency. Finally, FIG. 10 demonstrates the effect of molecular weight of the PSSA on antibody retention. The difference in PSSA molecular weight had little effect on antibody retention.

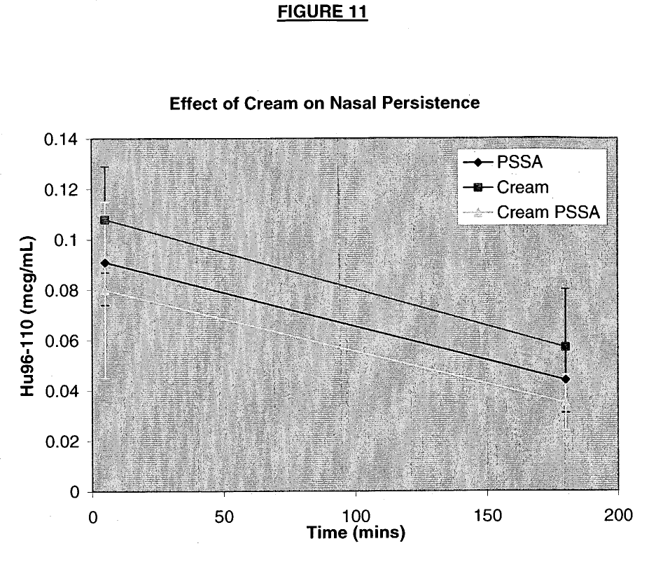

Mucoadhesive polymers may be combined with other compositions, such as cream formulations. As FIG. 11 demonstrates, cream formulations alone or in combination with mucoadhesive polymers prolong nasal retention of MAbs in a similar manner as mucoadhesive polymers alone.

The results of FIG. 6 demonstrate that mucoadhesive polymers prolong nasal retention of MAbs by many hours and thereby possibly decrease the frequency of repeat dosing necessary to block nasal colonization of S. aureus. The data presented in FIG. 6 represents the most effective formulations, 0.5% of 500,000 MW PSSA and 0.5% of 460,000 MW CS (PROTOSAN G-213). Since the method of delivery can effect nasal deposition and retention, various nasal spray devices will be evaluated for their influence on MAb delivery. Combinations of all these therapies will be examined to determine the optimal formulation for nasal MAb retention.

### Example 10

### Nasal Application of a Single Dose MAb in PSSA Alleviates Pre-Established Nasal Colonization

The efficacy of a MAb administered in a single dose in PSSA was tested in a cotton rat animal model for nasal S. aureus colonization. This is a variation of the mouse nasal colonization model previously described. Four to six week old Sigmadon hispidis cotton rats were used in this mode. At the same time, a Columbia agar plate containing 2% NaCl (CSA) was inoculated with S. aureus strain MBT 5040 from a frozen stock. MBT 5040 is a clinical MRSA strain isolated from tissue. This strain came from the Walter Reed Army Medical Center (WRAMC). The methicillin minimal inhibitory concentration (MIC) for MBT 5040 is >36 μg/ml. The MIC of a drug for a particular bacterial strain is the minimum concentration of the drug that inhibits normal growth of that particular bacterial strain. Growth on CSA plates encourages capsule formation around the bacteria, which in turn yields more efficient colonization of the nares.

On the day of instillation, S. aureus MBT 5040 was harvested from the CSA plate by scraping colonies into sterile PBS (1 ml/animal to be instilled) until the percent transmittance of the sample was approximately 10% at 650 nM. The bacteria were pelleted by centrifugation and then resuspended in 10 μl/animal of sterile PBS. Cotton rats were sedated with 200 μl of Ketamine (25 mg/kg), Rompun (2.5 mg/kg), and Acepromazine (2.5 mg/kg) delivered intramuscularly.

Ten microliters, 10⁹ S. aureus per animal, of MBT 5040 in PBS was instilled in the nares using a micropipette without touching the nares. Specifically, a drop of bacterial inoculum was placed on the nostril with a micropipettor, without touching the nose. The animal's regular process of respiration then inhaled the drop into the nares.

Two groups of five cotton rats each were instilled with 10⁹ S. aureus MBT 5040 as described above. Six days later, one group received a single nasal dose of 100 μg of A110 formulated in PSSA in a 20 μl volume. The PSSA-MAb suspension was prepared by mixing a 1% PSSA solution in PBS with an equal volume of 10 mg/mL A110 in PBS, so that the final concentrations of each component are 0.5% PSSA and 5 mg/mL A110. The two solutions were mixed together and immediately vortexed for 10 seconds, forming microparticles with a size around 0.5 μm to 10 μm on average.

Twenty four hours after instillation of the MAb in PSSA, the noses were harvested and checked for colonization. The animals were sacrificed by CO₂ inhalation. The noses were removed surgically, dissected, and vortexed well in 500 μl sterile PBS containing 0.5% Tween-20 to release colonizing bacteria. Fifty to 100 μl of PBS were plated on various types of agar plates to determine actual colonization. Specifically, because MBT 5040 S. aureus was nafcillin and streptomycin resistant, overall nasal colonization was measured as colony forming units (“CFUs”) on tryptic soy agar (TSA) plus 7.5% NaCl, nafcillin, and streptomycin plates. Microbiological tests were then used to determine which, if any, colonies on blood agar or TSA+7.5% NaCl were S. aureus.

As shown in Table 13, a single dose of A110 formulated in PSSA greatly reduced established nasal colonization in the cotton rat model.

TABLE 13

| Animal Number D | Untreated | A110 in PSSA |
|---|---|---|
| 1 | 610¹ | 99 |
| 2 | 1223¹¹ | 13 |
| 3 | 696¹¹ | 10 |
| 4 | 634¹¹ | 4 |
| 5 | 9¹¹ | 0 |
| Average | 634¹¹ | 25 |

¹1 CFUs recovered per nose.

### Example 11

### Human Antibodies That Bind LTA

Rather than humanizing a mouse antibody to minimize the HAMA response during treatment as described above, a skilled artisan can isolate a protective anti-LTA antibody that is fully human. There are a number of well-known alternative strategies one of ordinary skill in the art may use to produce completely human recombinant antibodies. One is the generation of antibodies using phage display technologies (79, 83). Specifically, human RNA is used to produce a cDNA library of antibody heavy and light chain fragments expressed on the surface of bacteriophage. These libraries can be used to probe against the antigen of interest (i.e., LTA) and the phage that bind, because of the antibody expressed on the surface, are then isolated. The DNA encoding the variable regions is sequenced and cloned for antibody expression.

Another method of producing human antibodies employs “humanized” mice. These transgenic mice have had their own antibody genes replaced with a portion of the human antibody gene complex so that upon inoculation with antigen, they produce human antibodies (77, 79, 80, 81, 83). The antibody producing cells that result can then be incorporated into the standard hybridoma technology for the establishment of specific monoclonal antibody producing cell lines.

Recombinant human antibodies are also produced by isolating antibody-producing B cells from human volunteers that have a robust anti-LTA response. Using fluorescence activated cell sorting (FACS) and fluorescently labeled LTA, cells producing the anti-LTA antibodies can be separated from the other cells. The RNA can then be extracted and the sequence of the reactive antibody variable regions determined (78, 82). The DNA sequence of the functional variable regions can be synthesized or cloned into mammalian expression vectors for large-scale human recombinant antibody production.

### CONCLUSION

Thus, Examples 3-5 show that the MAbs A110, MAb-11-232.3, and A110 Fc when instilled into mouse nares, blocked and/or alleviated colonization with S. aureus. Isotypes of these antibodies will also likely share this ability to block and/or alleviate colonization of S. aureus. Example 6 demonstrates that this effect is enhanced when the monoclonal antibody is conjugated to a carrier such as dextran. Example 7 demonstrates that it is possible to pre-instill MAb in the nares and still block nasal colonization in some mice. This example also demonstrates that pre-instillation of A110 and MAb-11-232.3 together may be better than instillation of either of the MAbs alone. Example 8 shows that two instillations of A110 in the nares of colonized mice alleviated the number of mice colonized at the end of the experiment, suggesting that nasal instillation of MAbs may be effective not only at blocking S. aureus nasal colonization but also at alleviating established nasal colonization. Example 9 shows that carriers can have an effect on the retention time of MAbs in the whole mouse nose. Example 10 shows that a single dose of A110 in PSSA administered to the nares of colonized cotton rats can effectively alleviate an established staphylococcal colonization.

Specific methods of delivery, specific dosing and timing for administration of antibodies will be performed to determine the most effective dose and schedule for blocking and/or clearing nasal colonization in mice. From this data, dosages and schedules will be developed for clinical trials in human subjects as well as other mammals.

One of skill in the art would realize that the monoclonal anti-staphylococcal antibodies which block staphylococcal nasal colonization are not limited to only those antibodies listed here and that the invention is also intended to include MAbs, and their isotypes, that bind to other antigens of S. aureus, including surface antigens, and MAbs to other bacteria that inhabit the mammalian nares. One of ordinary skill in the art would also recognize that these antibodies include MAbs with modified Fc regions. The usefulness of such other MAbs will be determined by comparison to a control group of mice treated with a chimerized anti-RSV monoclonal IgG antibody to ensure that antibodies specific for staphylococcal antigens cause the measured effect.

The following publications are hereby specifically incorporated by reference:

1. Aly, R. and S. Levit. 1987. Adherence of Staphylococcus aureus to squamous epithelium: role of fibronectin and teichoic acid. Rev. Infect. Dis. 9:S341-S350.

2. Aly, R., H. R. Shinefeld, C. Litz and H. I. Maibach. 1980. Role of teichoic acid in the binding of Staphylococcus aureus to nasal epithelial cells. J. Infect. Dis. 141:463-465.

3. Aly, R., H. I. Shinefield, W. G. Strauss and H. I. Maibach. 1977. Bacterial adherence to nasal mucosal cells. Infect. Immun. 17:546-549.

4. Anderson, I. and, D. F. Proctor. 1983. Measurement of nasal mucociliary clearance. Europ. J. Respir. Dis. 64:37-40.

5. Baddour, L. M., C. Lowrance, A. Albus, J. H. Lowrance, S. K. Anderson and J. C. Lee. 1992. Staphylococcus aureus microcapsule expression attenuates bacterial virulence in a rat model of experimental endocarditis. J. Infect. Dis. 165:749-753.

6. Beachy, E. H. and H. S. Courtney. 1987. Bacterial adherence: the attachment of group A streptococci to mucosal surfaces. Rev. Infect. Dis. 9:S475-S481.

7. Bibel, D. J., R. Aly, H. R. Shinefield, H. I. Maibach and W. G. Strauss. 1982. Importance of the keratinized epithelial cell in bacterial adherence. J. Invest. Dermatol. 79:250-253.

8. Braman, J., C. Papworth, and A. Greener. 1996. Site-directed mutagenesis using double-stranded plasmid DNA templates. Methods Mol Biol. 57:31-44.

9. Buckely, R. and R. Schiff. 1991. The use of intravenous immune globulin in immunodeficiency diseases. New Engl. J. Med. 325:110-115.

10. Carruthers, M. M. and W. J. Kabat. 1983. Mediation of staphylococcal adherence to mucosal cells by lipoteichoic acid. Infect. Immun. 40:444-446.

11. Chan, R. C. Y., G. Reid, R. T. Irvin, A. W. Bruce and J. W. Costerton. 1985. Competitive exclusion of uropathogens from human uroepithelial cells by Lactobacillus whole cell and cell wall fragments. Infect. Immun. 47:84-89.

12. Chang, F. Y., N. Singh, T. Gayowski, S. D. Drenning, M. M. Wagener and I. R. Marino. 1998. Staphylococcus aureus nasal colonization and association with infections in liver transplant recipients. Transplantation 65:1169-1172.

13. Chapoutot, C., G.-P. Pageaux, P. F. Perrigault, Z. Joomaye, P. Perney, H. Jean-Pierre, O. Jouquet, P. Blanc and D. Larrey. 1999. Staphylococcus aureus nasal carriage in 104 cirrhotic and control patients A prospective study. J. Hepatol. 30:249-253.

14. Chatfield, C., W. O'Neill, R. Cooke, K. McGhee, M. Issack, M. Rahman and W. Noble. 1994. Mupirocin-resistant Staphylococcus aureus in a specialist school population. J. Hosp. Infect. 26:273-278.

15. Chugh, T. D., G. J. Burns, H. J. Shuhaiber and G. M. Bahr. 1990. Adherence of Staphylococcus epidermidis to fibrin-platelet clots in vitro mediated by lipoteichoic acid. Infect. Immun. 58:315-319.

16. Chugh, T. D., G. J. Burns, H. J. Shuhaiber and E. A. Bishbishi. 1989. Adherence of Staphylococcus epidermis to human epithelial cells: evidence for lipase-sensitive adhesion and glycoprotein receptor. Current Micro. 18:109-112.

17. Cookson, B. 1990. Mupirocin resistance in staphylococci. J. Antimicrob. Chemother. 25:497-503.

18. Corbella, X., M. Dominguez, M. Pujol, J. Aytas, M. Sendra, R. Pallares, J. Ariza and F. Gudiol. 1997. Staphylococcus aureus nasal carriage as a marker for subsequent staphylococcal infections in intensive care units patients. Eur. J. Clin. Microbial. Infect. Dis. 16:351-357.

19. Dawson, S., L. Finn, J. McCulloch, S. Kilvington and D. Lewis. 1994. Mupirocin-resistant MRSA. J. Hosp. Infect. 28:75-78.

20. De Kimpe, S. J., M. Kengatharan, C. Thiemermann and J. R. Vane. 1995. The cell wall components peptidoglycan and lipoteichoic acid from Staphylococcus aureus act in synergy to cause shock and multiple organ failure. PNAS-USA 92:10359-10363.

21. Doebbeling, B. N., D. Breneman, H. Neu, R. Aly, B. Yangco, H. Holley, R. Marsh, M. Pfaller, J. McGowan, B. Scully, D. Reagan and R. Wenzel. 1993. Elimination of Staphylococcus aureus nasal carriage in health care workers: analysis of six clinical trials with calcium mupirocin ointment. Clin. Infect. Dis. 17:466-474.

22. Fierobe, L., D. Decre, C. Muller, J.-C. Lucet, J.-P. Marmuse, J. Mantz and J.-M. Demonts. 1999. Methicillin-resistant Staphylococcus aureus as a causative agent of postoperative intra-abdominal infection: relation to nasal colonization. Clin. Infect. Dis. 29:1231-1238.

23. Foster, T. J. and D. McDevitt. 1994. Surface-associated proteins of Staphylococcus aureus: their possible role in virulence. FEMS Microbiol. Lett. 118:199-205.

24. Frebourg, N., B. Cauliez and J.-F. Lemeland. 1999. Evidence for nasal carriage of methicillin-resistant staphylococci colonizing intravascular devices. J. Clin. Micro. 37:1182-1185.

25. Granato, D., F. Perotti, I. Masserey, M. Rouvet, M. Golliard, A. Servin and D. Brassart. 1999. Cell surface-associated lipoteichoic acid acts as an adhesion factor for attachment of Lactobacillus johnsonii La1 to human enterocyte-like Caco-2 cells. Appl. Enviro. Micro. 65:1071-1077.

26. Herrmann, M., J. Hartleib, B. Kehrel, R. R. Montgomery, J. J. Sixma and G. Peters. 1997. Interaction of von Willebrand factor with Staphylococcus aureus. J. Infect. Dis. 176:984-991.

27. Herrmann, M., Q. J. Lai, R. M. Albrecht, D. F. Mosher and R. A. Proctor. 1993. Adhesion of Staphylococcus aureus to surface-bound platelets: role of fibrinogen/fibrin and platelet integrins. J. Infect. Dis. 167:312-322.

28. Hoefnagels-Schuermans, A., W. E. Petermans, M. Jorissen, S. Van Lierde, J. van den Oord, R. De Vos and J. Van Eldere. 1999. Staphylococcus aureus adherence to nasal epithelial cells in a physiological in vitro model. In Vitro Cell. Dev. Biol.-Animal. 35:472-480.

29. Johnson S, C. Oliver, G. A. Prince, V. G. Hemming, D. S. Pfarr, S. C. Wang, M. Dormitzer, J. O'Grady, S. Koenig, J. K. Tamura, R. Woods, G. Bansal, D. Couchenour, E. Tsao, W. C. Hall, and J. F. Young. 1997 Development of a humanized monoclonal antibody (Medi 493) with potent in vitro and in vivo activity against respiratory syncytial virus. J. Infect Dis. 176:1215-1224.

30. Kiser, K. B., J. M. Cantey-Kiser and J. C. Lee. 1999. Development and characterization of a Staphylococcus aureus nasal colonization model in mice. Infect. Immun. 67:5001-5006.

31. Kluytmans, J., A. van Belkum and H. Verbrugh. 1997. Nasal carriage of Staphylococcus aureus: epidemiology, underlying mechanisms, and associated risks. Clin. Micro. Rev. 10:505-520.

32. Kluytmans, J. A., J. W. Mouton, M. VandenBergh, M.-J. Manders, A. Maat, J. Wagenvoort, M. Michel and H. Verbrugh. 1996. Reduction of surgical-site infections in cardiothoracic surgery by elimination of nasal carriage of Staphylococcus aureus. Infect. Control. Hosp. Epidem. 17:780-785.

33. Kublick, H. and M. T. Vidgren. 1998. Nasal delivery systems and their effect on deposition and absorption. Advan. Drug Deliv. Rev. 29:157-177.

34. Lee, Y.-L., T. Cesario, A. Pax, C. Tran, A. Ghouri and L. Thrupp. 1999. Nasal colonization by Staphylococcus aureus in active, independent community seniors. Age Ageing. 28:229-232.

35. Lees A; F. Finkelman; J. K. Inman; K. Witherspoon; P. Johnson; J. Kennedy; and J. J. Mond. 1994. Enhanced immunogenicity of protein-dextran conjugates: I. Rapid stimulation of enhanced antibody responses to poorly immunogenic molecules. Vaccine 12:1160-1166.

36. Ma, J. K.-C., M. Hunjan, R. Smith, C. Kelly and T. Lehner. 1990. An investigation into the mechanism of protection by local passive immunization with MAbs against Streptococcus mutans. Infect. Immun. 58:3407-3414.

37. Marples, R. R., D. C. E. Speller and B. D. Cookson. 1995. Prevalence of mupirocin resistance in Staphylococcus aureus. J. Hosp. Infect. 29:153-155.

38. Martin, J., F. Perdreau-Remington, M. Kartalija, O. Pasi, M. Webb, J. Gerberding, H. Chambers, M. Tauber and B. Lee. 1999. A randomized clinical trial of mupirocin in the eradication of Staphylococcus aureus nasal carriage in human immunodeficiency virus disease. J. Infect. Dis. 180:896-899.

39. Mazenec, M. B., C. S. Kaetzel, M. E. Lamm, D. Fletcher, J. Peterra and J. G. Nedrud. 1995. Intracellular neutralization of Sendai and influenza viruses by IgA MAbs. Adv. Exp. Med. Biol. 371A:651-654.

40. McDevitt, D., P. Francois, P. Vaudaux and T. Foster. 1994. Molecular characterization of the clumping factor (fibrinogen receptor) of Staphylococcus aureus. Mol. Microbiol. 11:237-248.

41. Merkus, F. W., J. C. Verhoef, N. G. Schipper, and E. Marttin. 1999. Cyclodextrins in nasal drug delivery. Advan. Drug Deliv. Rev. 36:41-57.

42. Mest, D. R., D. H. Wong, K. J. Shimoda, M. E. Mulligan and S. E. Wilson. 1994. Nasal colonization with methicillin-resistant Staphylococcus aureus on admission to surgical intensive care unit increases the risk of infection. Anesth. Analg. 78:644-650.

43. Mosher, D. F. and R. A. Proctor. 1980. Binding and factor XIII-mediated cross linking of a 27-kilodalton fragment of fibronectin to Staphylococcus aureus. Science 209:927-929.

44. Nakamura, K. et al. 1999. Uptake and Release of Budesonide from Mucoadhesive, pH-sensitive Copolymers and Their Application to Nasal Delivery. J. Control. Release 61:329-335.

45. Natsume, H., S. Iwata, K. Ohtak, M. Miyamoto, M. Yamaguchi, K. Hosoya, and D. Kobayashi. 1999. Screening of cationic compounds as an absorption enhancer for nasal drug delivery. Int. J Pharma. 185:1-12.

46. Nealon, T. J. and S. J. Mattingly. 1984. Role of cellular lipoteichoic acids in mediating adherence of serotype III strains pf group B streptococci to human embryonic, fetal and adult epithelial cells. Infect. Immun. 43:523-530.

47. Nelson, M. and M. McClelland. 1992. Use of DNA methyltransferase/endonuclease enzyme combinations for megabase mapping of chromosomes. Methods Enzymol. 216:279-303.

48. Nguyen, M. H., C. Kauffman, R. Goodman, C. Squier, R. Arbeit, N. Singh, M. Wagener and V. Yu. 1999. Nasal carriage of and infection with Staphylococcus aureus in HIV-infected patients. Ann. Int. Med. 130:221-225.

49. Olmsted, S. and N. Norcross. 1992. Effect of specific antibody on adherence of Staphylococcus aureus to bovine mammary epithelial cells. Infect. Immun. 60:249-256.

50. Patrick, C. C., M. R. Plaunt, S. V. Hetherington and S. M. May. 1992. Role of the Staphylococcus epidermidis slime layer in experimental tunnel tract infections. Infect. Immun. 60:1363-1367.

51. Pei, L., M. Palma, M. Nilsson, B. Guss and J.-l. Flock. 1999. Functional studies of fibrinogen binding protein from Staphylococcus epidermidis. Infect. Immun. 67:4525-4530.

52. Pennington, J. 1990. Newer uses for intravenous immunoglobulins as anti-infective agents. Antimicrob. Agents Chemother. 34:1463-1466.

53. Procter, R. A., R. J. Hamill, D. F. Mosher, J. A. Textor and P. J. Olbrantz. 1983. Effects of subinhibitory concentrations of antibiotics on Staphylococcus aureus interactions with fibronectin. J. Antimicro. Chemo. 12:85-95.

54. Ramisse, F., M. Szatanik, P. Binder and J.-M. Alonso. 1993. Passive local immunotherapy of experimental staphylococcal pneumonia with human intravenous immunoglobulin. J. Infect. Dis. 168:1030-1033.

55. Ramkissoon-Ganorkar, C. et al. 1999. Modulating insulin-release profile from pH/thermosensivite polymeric beads through polymer molecular weight. J. Contr. Release 59:287-298.

56. Remington's Pharmaceutical Sciences, 18th Edition (A. Gennaro, ed., Mack Pub., Easton, Pa., 1990).

57. Ribeiro, A. J. et al. 1999. Microencapsulations of lipophilic drugs in chitosan-coated alginate microspheres. Int. J Pharm. 187:115-123.

58. Sambrook et al. 1989. Molecular Cloning, A Laboratory Manual, 2^{nd} Ed., Cold Spring Harbor Press, Cold Spring Harbor, N.Y.

59. Sanford, B. A., V. L. Thomas and M. A. Ramsay. 1989. Binding of staphylococci to mucus in vivo and in vitro. Infect. Immun. 57:3735-3742.

60. Schennings, T., A. Heimdahl, K. Coster and J. Flock. 1993. Immunization with fibronectin binding protein from Staphylococcus aureus protects against experimental endocarditis in rats. Microb. Pathog. 15:227-236.

61. Schwab, U. E., A. E. Wold, J. L. Carson, M. W. Leigh, P.-W. Cheng, P. H. Gilligan and T. F. Boat. 1993. Increased adherence of Staphylococcus aureus from cystic fibrosis lungs to airway epithelial cells. Am. Rev. Respir. Dis. 148:365-369.

62. Soane, R. J. et al. 1999. Evaluation of the clearance characteristics of bioadhesive systems in humans. Int. J. Pharm. 178:55-65.

63. Soto, N., A. Vaghjimal, A. Stahl-Avicolli, J. Protic, L. Lutwick and E. Chapnick. 1999. Bacitracin versus mupirocin for Staphylococcus aureus nasal colonization. Infect. Cont. Hosp. Epidem. 20:351-353.

64. Suzuki, Y. and Y. Makino. 1999. Mucosal drug delivery using cellulose derivative as a functional polymer. J. Control. Release. 62:101-107.

65. Takenaga, M., Y. Sirizawa, Y. Azechi, A. Ochiai, Y. Kosaka, R. Igarashi, and Y. Mizushima. 1998. Microparticle resins as a potential nasal drug delivery system for insulin. J. Control. Release. 52:81-87.

66. Teti, G., M. S. Chiofalo, F. Tomasello, C. Fava and P. Mastroeni. 1987. Mediation of Staphylococcus saprophyticus adherence to uroepithelial cells by lipoteichoic acid. Infect. Immun. 55:839-842.

67. VadenBergh, M., E. Yzerman, A. Van Belkum, H. Boelens, M. Simmons and H. Verbrugh. 1999. Follow-up of Staphylococcus aureus nasal carriage after 8 years: redefining the persistent carrier state. J. Clin. Micro. 37:3133-3140.

68. Vaudaux, P., E. Huggler, P. Lerch, J. Morgenthaler, U. Nydegger, F. Schumacher-Perdreau, P. Lew and F. Waldvogel. 1989. Inhibition by immunoglobulins of Staphylococcus aureus adherence to fibronectin-coated foreign surfaces. J. Invest. Surg. 2:397-408.

69. Ward, T. T. 1992. Comparison of in vitro adherence of methicillin-sensitive and methicillin-resistant Staphylococcus aureus to human nasal epithelial cells. J. Infect. Dis. 166:400-404.

70. White, A. and J. Smith. 1963. Nasal reservoir as the source of extranasal staphylococci. Antimicrob. Agent. Chem. 3:679-683.

71. Yano, M., Y. Doki, M. Inoue, T. Tsujinaka, H. Shiozaki and M. Monden. 2000. Preoperative intranasal mupirocin ointment significantly reduces postoperative infection with Staphylococcus aureus in patients undergoing upper gastrointestinal surgery. Surg. Today (Japan). 30:16-21.

72. Yokoyama, Y., Y. Harabuchi, H. Kodama, H. Murakata and A. Kataura. 1996. Systemic immune response to Streptococcal and Staphylococcal lipoteichoic acids in children with recurrent tonsillitis. Acta Otolaryngol. Suppl. (Norway). 523:108-111.

73. Winter, G., A. D. Griffiths, et al. (1994). “Making antibodies by phage display technology.” Annu Rev Immunol 12: 433-55

73. Yu, V. L., A. Goetz, M. Wagener, P. B. Smith, J. D. Rihs, J. Hanchett and J. J. Zuravleff. 1986. Staphylococcus aureus nasal carriage and infection in patients on hemodialysis. New Engl. J. Med. 315:91-96.

74. Ausubel et al. (ed.) 1989. Current Protocols in Molecular Biology, John Wiley & Sons.

75. Borrebaeck, Carl A. K. 1995. Antibody Engineering, 2^{nd} Ed., Oxford University Press, NY.

76. Harlow, Ed; Lane, David. 1988. Antibodies: A Laboratory Manual, Cold Spring Harbor Press, Cold Spring Harbor, N.Y.

77. Green, L. L., M. C. Hardy, et al. (1994). “Antigen-specific human monoclonal antibodies from mice engineered with human Ig heavy and light chain YACs.” Nat Genet 7(1): 13-21.

78. Kantor, A. B., C. E. Merrill, et al. (1995). “Development of the antibody repertoire as revealed by single-cell PCR of FACS-sorted B-cell subsets.” Ann N Y Acad Sci 764: 224-7.

79. Low, N. M., P. H. Holliger, et al. (1996). “Mimicking somatic hypermutation: affinity maturation of antibodies displayed on bacteriophage using a bacterial mutator strain.” J Mol Biol 260(3): 359-68.

80. Wagner, S. D., A. V. Popov, et al. (1994). “The diversity of antigen-specific monoclonal antibodies from transgenic mice bearing human immunoglobulin gene miniloci.” Eur J Immunol 24(11): 2672-81.

81. Wagner, S. D., G. T. Williams, et al. (1994). “Antibodies generated from human immunoglobulin miniloci in transgenic mice.” Nucleic Acids Res 22(8): 1389-93.

82. Wang, X. and B. D. Stollar (2000). “Human immunoglobulin variable region gene analysis by single cell RT-PCR.” J Immunol Methods 244(1-2): 217-25.

83. Winter, G., A. D. Griffiths, et al. (1994). “Making antibodies by phage display technology.” Annu Rev Immunol 12: 433-55.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for treating a patient, comprising instilling in to the nares of a patient, an effective amount of a composition comprising at least one MAb that specifically binds at least one antigen of staphylococci;
wherein treatment results in
a) no nasal colonization by staphylococci for at least 12 hours after administration, or
b) a decrease in the number of staphylococcal colonies in the nares, or
c) a decrease in the frequency of positive cultures taken from the nares, or
d) a decrease in the frequency of staphylococcal infections.

2. The method of claim 1, wherein the composition comprises a multiplicity of MAbs having non-identical amino acid sequences.

3. The method of claim 1, further comprising the instillation of at least one anti-staphylococcal drug.

4. The method of claim 3, wherein the anti-staphylococcal drug is selected from lysostaphin and nisin.

5. The method of claim 1, wherein at least one MAb specifically binds to a staphylococcal surface antigen.

6. The method of claim 1, wherein at least one MAb that specifically binds to LTA.

7. The method of claim 1, wherein at least one MAb specifically binds to peptidoglycan.

8. The method of claim 1, wherein at least one MAb specifically binds to a staphylococcal surface antigen selected from virulence antigens and adherence antigens.

9. The method of claim 1, wherein the composition is instilled in a form selected from drops, spray, powder, aerosol, mist, gel, lotion, cream, paste, particulate, or pellet.

10. The method of claim 1, wherein the composition comprises a pharmaceutically acceptable carrier.

11. The method of claim 1, wherein the composition comprises a mucoadhesive.

12. The method of claim 1, wherein the composition comprises a multiplicity of MAb molecules are bound to a carrier selected from molecules, polymers, and particles.

13. The method of claim 1, wherein the composition comprises microspheres containing or bearing said at least one MAb.

14. The method of claim 1, wherein the composition comprises a carrier, wherein said carrier microencapsulates at least one MAb.

15. The method of claim 1, wherein the composition comprises a carrier selected from natural polymers, semi-synthetic polymers, synthetic polymers, and liposomes.

16. The method of claim 1, wherein the composition comprises a carrier selected from polyphosphoesters, dendrimers, polyethylene glycol, poly (lactic acid), polystyrene sulfonate, and poly (lactide coglycolide), chitosan, hydroxypropyl cellulose, proteins, or polysaccharides.

17. The method of claim 1 wherein the composition comprises chitosan.

18. The method of claim 1, wherein the composition comprises polystyrene sulfonate.

19. The method of claim 1, wherein the composition comprises a polysaccharide covalently conjugated to said at least one MAb.

20. The method of claim 1, wherein at least one MAb is selected from chimeric and humanized MAbs.

21. The method of claim 1, wherein at least one monoclonal antibody is human.

22. The method of claim 1, wherein at least one MAb is selected from A110, A110 Fc, MAb-11-232.3, MAb-11-248.2, MAb-11-569.3, A120, and 99-110FC12 IE4.

23. The method of claim 1, wherein at least one MAb comprises a human heavy chain constant region selected from IgG, IgA, and IgM.

24. The method of claim 1, wherein at least one MAb comprises an IgG1 human heavy chain constant region.

25. The method of claim 1, wherein at least one MAb comprises amino acid sequence of SEQ ID NO: 1

26. The method of claim 1, wherein at least one MAb contains a modified Fc portion.

27. The method of claim 26, wherein the modification reduces nonspecific binding of the MAb via the Fc portion.

28. The composition of claim 32, wherein at least one MAb is selected from a Fab, Fab′, F(ab′)2, Fv, SFv, and scFv.

29. A method for treating a patient, comprising applying to the previously colonized epithelial surface of a patient, an effective amount of a composition comprising at least one MAb that specifically binds at least one antigen of staphylococci;
wherein treatment results in
a) a decrease in staphylococcal colonization of the epithelial surface treated, or
b) a discernable decrease in the frequency of staphylococcal infections.

30. The method of claim 29, wherein the previously colonized epithelium is selected from the nose, the skin, the eyes, the mouth, and the respiratory track.

31. The method of claim 30, wherein the previously colonized epithelium is the anterior nares of the nose.

32. A composition comprising at least one MAb that specifically binds at least one antigen of staphylococci and a mucoadhesive carrier;
wherein treatment of a patient with said composition by nasal instillation results in
a) no nasal colonization by staphylococci for at least 12 hours after administration, or
b) a discernable decrease in the number of staphylococcal colonies in the nares, or
c) a discernable decrease in the frequency of positive cultures taken from the nares, or
d) a discernable decrease in the frequency of staphylococcal infections.

33. The composition of claim 32, wherein at least one MAb is microencapsulated.

34. The composition of claim 32, wherein the mucoadhesive carrier comprises chitosan.

35. The composition of claim 32, wherein the mucoadhesive carrier comprises polystyrene sulfonate.

36. The composition of claim 32, wherein the mucoadhesive carrier comprises hydroxypropyl cellulose.

37. The composition of claim 32, wherein at least one MAb is selected from chimeric and humanized MAbs.

38. The composition of claim 32, wherein at least one MAb is human.

39. The composition of claim 32, wherein at least one MAb is selected from a Fab, Fab′, F(ab′)₂, Fv, SFv, and scFv.
